(19) United States
(12) Patent Application Publication
(10) Pub. No.: US 2016/0228567 A1
(43) Pub. Date: Aug. 11, 2016

(54) Hypoxia-Targeted Delivery System for Pharmaceutical Agents

(71) Applicant: NORTHEASTERN UNIVERSITY, Boston, MA (US)

(72) Inventors: Vladimir TORCHILIN, Charlestown, MA (US); Swati BISWAS, Boston, MA (US); Federico PERCHE, Boston, MA (US)

(21) Appl. No.: 15/022,993

(22) PCT Filed: Oct. 21, 2014
(86) PCT No.: PCT/US14/61582

§ 371 Date: Mar. 18, 2016

Related U.S. Application Data
(60) Provisional application No. 61/893,472, filed on Oct. 21, 2013.

(51) Int. Cl. A61K 47/48; A61K 45/06; C12N 15/113; A61K 31/713; A61K 31/7088

(52) U.S. Cl. CPC A61K 47/48215; A61K 47/4803; A61K 47/48023; A61K 47/48192; A61K 47/48053; A61K 31/713; A61K 31/7088; C12N 15/113; A61K 47/48807; A61K 45/06; C12N 2310/14; C12N 2310/11; C12N 2310/351; C12N 2320/32; C12N 2320/31

(57) ABSTRACT

Molecular compositions, nanoparticle compositions, and pharmaceutical compositions of the invention provide for the delivery of a polynucleotide to a hypoxic cell or tissue. The compositions can also be used for the delivery a hydrophobic pharmaceutical agent, either alone or in combination with a polynucleotide, to a hypoxic cell or tissue. Methods of making such compositions and methods of using such composition to treat a condition associated with a hypoxic cell or tissue are provided as well. Also provided are kits for use in treating a condition associated with a hypoxic cell or tissue.

### CROSS REFERENCE TO RELATED APPLICATIONS

This application claims the benefit of U.S. Provisional Application No. 61/893,472, filed Oct. 21, 2013 and entitled “Hypoxia-Targeted Delivery of Pharmaceutical Agents,” which is hereby incorporated by reference in its entirety.

### STATEMENT REGARDING FEDERALLY SPONSORED RESEARCH OR DEVELOPMENT

This invention was developed with financial support from Grant No. U54CA151881 from the National Institutes of Health. The U.S. Government has certain rights in the invention.

### BACKGROUND

The specificity and potency of siRNA regulation of gene expression holds great promise for cancer therapy. [5] However, siRNA delivery to hypoxic regions is challenging since such regions are distant from blood vessels and have increased efflux transporters. [1] In addition, the use of nanocarriers is required to protect siRNA from degradation and to promote its cellular internalization and endosomal escape. [5a] Usually, nanoparticle applications rely on the enhanced permeability and retention (EPR) effect for accumulation in tumor tissue. [6] Nanoparticles are expected to show preferential extravasation from the circulation when they reach the altered tumor vasculature with its widened endothelial fenestrae and deficient pericyte coverage. Conjugation of polyethyleneglycol (PEG) to nanoparticles extends their blood circulation time, increasing the probability of tumor accumulation by EPR. [7] However, PEGylation can also hinder cellular uptake resulting in decreased therapeutic activity. [5a, 7a] This PEG dilemma led to the design of nanoformulations with PEG that can be detached upon tumor stimulus to target payload delivery. [6, 8] Nitroimidazole derivatives have been proposed as hypoxia sensors since they are subject to intracellular reduction with formation of free radicals. [1a, 2b, 4b] While these free radicals are rapidly oxidized by molecular oxygen, their stabilization under hypoxia leads to reduction-mediated cleavage. [1b, 4a,b, 9] Nagano and co-workers demonstrated successful hypoxia imaging in vivo with azobenzene-based probes. [4a, 9].

### SUMMARY OF THE INVENTION

Described herein are molecular compositions, nanoparticle compositions, and pharmaceutical compositions for the delivery of a polynucleotide to a hypoxic cell or tissue. The compositions can also be used for the delivery a hydrophobic pharmaceutical agent, alone or in combination with a polynucleotide, to a hypoxic cell or tissue. Methods of making such compositions and methods of using such composition to treat a condition associated with a hypoxic cell or tissue are provided as well. Also described are kits for use in treating a condition associated with a hypoxic cell or tissue.

In one aspect, the invention is a hypoxia-sensitive polynucleotide-binding molecule including: an uncharged hydrophilic polymer; an azobenzene moiety, wherein the azobenzene moiety is attached to the to the uncharged hydrophilic polymer by a first covalent linkage; a positively-charged polymer, wherein the positively-charged polymer is attached to the azobenzene moiety by a second covalent linkage, and wherein the positively-charged polymer binds one or more polynucleotide molecules; and a phospholipid, wherein the phospholipid is attached to the positively-charged polymer by a third covalent linkage; wherein the uncharged hydrophilic polymer, the azobenzene moiety, the positively-charged polymer, and the phospholipid are present in the molecule in about a 1:1:1:1 molar ratio.

In some embodiments, the uncharged polymer may be polyethylene glycol, polyvinylpyrrolidone, or polyacrylamide. In an embodiment, the uncharged polymer is polyethylene glycol. In an embodiment, the polyethylene glycol has an average molecular weight from about 1000 to about 5000 daltons. In an embodiment, the polyethylene glycol has an average molecular weight of about 2000 daltons.

In some embodiments, the azobenzene moiety is, or is derived from, azobenzene-4,4′-dicarboxamide.

In some embodiments, the positively-charged polymer may be polyethylenimine, polylysine, a cationic peptide, poly(dl-lactide-co-glycolide), poly(amidoamine), or poly(propylenimine). In an embodiment, the positively-charged polymer is polyethylenimine. In an embodiment, the polyethylenimine has a molecular weight from about 500 daltons to about 5000 daltons. In an embodiment, the polyethylenimine has an average molecular weight of about 1800 daltons. In an embodiment, the polyethylenimine has a branched structure.

In some embodiments, the phospholipid may be phosphatidic acid, phosphatidylethanolamine, phosphatidylcholine, phosphatidylserine, phosphatidylinositol, phosphotidylglycerol, or a sphingolipid. In some embodiments, the phospholipid comprises fatty acid side chains each having from 12-20 carbon atoms. In some embodiments, the fatty acid side chains are saturated, monounsaturated, diunsaturated, or triunsaturated. In some embodiments, the phospholipid is phosphtatidylethanolamine. In an embodiment, the phosphatidylethanolamine is 1,2-dioleoyl-sn-glycero-3-phosphoethanolamine.

In some embodiments, the covalent linkages may be peptide bonds, amide bonds, ester bonds, ether bonds, alkyl bonds, carbonyl bonds, alkenyl bonds, thioether bonds, disulfide bonds, and/or azide bonds. In some embodiments, each covalent linkages is a peptide bond.

In one aspect, the invention is a nanoparticle composition for delivery of a polynucleotide to a hypoxic cell or tissue, and the composition includes a plurality of hypoxia-sensitive polynucleotide-binding molecules suspended in an aqueous medium and aggregated to form one or more nanoparticles.

In some embodiments, the nanoparticle composition includes one or more polynucleotides that are non-covalently bound to the positively-charged polymers of the hypoxia-sensitive polynucleotide-binding molecule. In some embodiments, the polynucleotide(s) is single-stranded RNA, double-stranded RNA, single-stranded DNA, or double-stranded RNA. In some embodiments, the polynucleotide(s) is siRNA. In some embodiments, the polynucleotides are two or more different species of siRNA. In some embodiments, the polynucleotide is an antisense oligonucleotide. In some embodiments, the polynucleotide targets the expression of one or more of survivin, Eg5, EGFR, XIAP, CDC45L, SUV420h1, WEE1, HDAC2, RBX 1, CDK4, CSN5, FOXM1, R1 (RAM2), LSD1, CSTF2, Nectin-4, ERCC6L, PKIB, NAALADL2, PRMT1, COPZ1, SYNGR4, P-glycoprotein, VEGFR, and VEGF.

In some embodiments, the nanoparticle composition has a nitrogen:phosphate ratio from about 1:5 to about 1:50.

In some embodiments, the nanoparticles are micelles. In some embodiments, the micelles have a worm-like morphology (i.e., exhibiting a long, flexible structure). In some embodiments the micelles have an average diameter from about 10 to about 50 nm.

In some embodiments, the hypoxic cell or tissue is associated with cancer. In some embodiments, the cancer is associated with a solid tumor. In some embodiments, the cancer may be uterine cancer, cervical cancer, prostate cancer, ovarian cancer, sarcoma, or head and neck cancer.

In some embodiments, the azobenzene moiety of the hypoxia-sensitive polynucleotide-binding molecules is cleavable in a hypoxic environment. In some embodiments, cleavage of the azobenzene moiety causes release of the uncharged hydrophilic polymers from the nanoparticles. In some embodiments, cleavage of the azobenzene moiety results in increased cellular uptake of polynucleotides bound to the positively-charged polymers of the hypoxia-sensitive polynucleotide-binding molecules.

In some embodiments, the nanoparticle composition includes a hydrophobic pharmaceutical agent. In some embodiments, the hydrophobic pharmaceutical agent is an anti-cancer agent. In some embodiments, the anti-cancer agent may be altretamine, aminoglutethimide, amsacrine (m-AMSA), azacitidine, baccatin III, bleomycin, busulfan, carmustine (BCNU), chlorambucil, cytarabine HCl, dacarbazine, dactinomycin, daunorubicin, docetaxel, doxorubicin, etoposide (VP-16), 5-fluorouracil, floxuridine, flutamide, hydroxyurea, ifosfamide, leuprolide acetate, lomustine (CCNU), melphalan, methotrexate, mitomycin, mitotane (o.p'-DDD), octreotide, paclitaxel, pentostatin, plicamycin, procarbazine HCl, semustine (methyl-CCNU), streptozocin, tamoxifen citrate, teniposide (VM-26), thioguanine, thiotepa, vindesine, vinblastine, vincristine sulfate, or a combination thereof.

In some embodiments, the nanoparticle composition consists only of a plurality of hypoxia-sensitive polynucleotide-binding molecules.

In one aspect, the invention is a pharmaceutical composition that includes a nanoparticle composition of the invention suspended in an aqueous buffer.

In some embodiments, the pharmaceutical composition includes an excipient. For example, the excipient may be a buffer, electrolyte, or other inert component.

In one aspect, the invention is a method of making a hypoxia-sensitive polynucleotide-binding molecule from an uncharged hydrophilic polymer having a first reactive group, an azobenzene derivative having a second reactive group and a third reactive group, a positively-charged polymer having a fourth reactive group and a fifth reactive group, and a phospholipid having a sixth reactive group, the method including the steps of: reacting the first reactive group on the uncharged hydrophilic polymer with the second reactive group on the azobenzene derivative, wherein the uncharged hydrophilic polymer and the azobenzene derivative are present in about a 1:1 molar ratio, to create a covalent linkage between the uncharged hydrophilic polymer and the azobenzene derivative; reacting the third reactive group on the azobenzene derivative with the fourth reactive group on the positively-charged polymer, wherein the azobenzene derivative and the positively-charged polymer are present in about a 1:1 molar ratio, to create a covalent linkage between the azobenzene derivative and the positively-charged polymer; and reacting the fifth reactive group on the positively-charged polymer with the sixth reactive group on the phospholipid, wherein the positively charged polymer and the phospholipid are present in about a 1:1 molar ratio, to create a covalent linkage between the positively-charged polymer and the phospholipid.

The steps of the method of making the hypoxia-sensitive polynucleotide-binding molecule can be performed in any order. In one embodiment, the uncharged hydrophilic polymer and azobenzene derivative are reacted first, the azobenzene derivative and positively-charged polymer are reacted second, and the positively-charged polymer and phospholipid are reacted third. In one embodiment, the uncharged hydrophilic polymer and azobenzene derivative are reacted first, the positively-charged polymer and phospholipid are reacted second, and the azobenzene derivative and positively-charged polymer are reacted third. In one embodiment, the azobenzene derivative and positively-charged polymer are reacted first, the uncharged hydrophilic polymer and azobenzene derivative are reacted second, and the positively-charged polymer and phospholipid are reacted third. In one embodiment, the azobenzene derivative and positively-charged polymer are reacted first, the positively-charged polymer and phospholipid are reacted second, and the uncharged hydrophilic polymer and azobenzene derivative are reacted third. In one embodiment, the positively-charged polymer and phospholipid are reacted first, the uncharged hydrophilic polymer and azobenzene derivative are reacted second, and the azobenzene derivative and positively-charged polymer are reacted third. In one embodiment, the positively-charged polymer and phospholipid are reacted first, the azobenzene derivative and positively-charged polymer are reacted second, and the uncharged hydrophilic polymer and azobenzene derivative are reacted third.

In some embodiments, the uncharged hydrophilic polymer is polyethylene glycol 2000-N-hydroxysuccinamide ester.

In some embodiments, the azobenzene derivative is azobenzene-4,4′-dicarboxylic acid.

In some embodiments, the positively-charged polymer is branched polyethylenimine having an average molecular weight of about 1800 daltons.

In some embodiments, the phosphatidylethanolamine is 1,2-dioleoyl-sn-glycero-3-phosphoethanolamine-N-(glutaryl).

In some embodiments, the uncharged hydrophilic polymer and azobenzene derivative are reacted in the presence of N-(3-dimethylaminopropyl)N′-ethylcarbodiimide hydrochloride, N-hydroxysuccinimide, pyridine, and 4-dimethylaminopyridine at room temperature.

In some embodiments, the azobenzene derivative and positively-charged polymer are reacted in the presence of N-(3-dimethylaminopropyl)N′-ethylcarbodiimide hydrochloride, N-hydroxysuccinimide, triethylamine, and CHCl₃ at room temperature.

In some embodiments, the positively-charged polymer and phospholipid are reacted in the presence of N-(3-dimethylaminopropyl)N′-ethylcarbodiimide hydrochloride, N-hydroxysuccinimide, triethylamine, and CHCl₃ at room temperature.

In one aspect, the invention is a method of making a nanoparticle composition including the hypoxia-sensitive polynucleotide-binding molecule, the method including the steps of: providing a solution of the hypoxia-sensitive polynucleotide-binding molecule in a non-aqueous solvent; and replacing the non-aqueous solvent with an aqueous medium to form an aqueous suspension comprising nanoparticles, the nanoparticles comprising aggregates of a plurality of the hypoxia-sensitive polynucleotide-binding molecules.

The non-aqueous solvent may be replaced with an aqueous medium by any method. In some embodiments, the non-aqueous solvent is removed by dialyzing the solution of the hypoxia-sensitive polynucleotide-binding molecule against an aqueous medium. In some embodiments, the non-aqueous solvent is removed by evaporating the non-aqueous solvent to form a dry film of the hypoxia-sensitive polynucleotide-binding molecule and suspending the dry film of said molecule in an aqueous medium.

In some embodiments, the method includes the step of adding a hydrophobic pharmaceutical agent to the solution of the hypoxia-sensitive polynucleotide-binding molecule in a non-aqueous solvent, whereby the nanoparticles produced by replacing the non-aqueous solvent with an aqueous medium contain the hydrophobic pharmaceutical agent.

In some embodiments, the method includes the step of adding a hydrophobic pharmaceutical agent to the aqueous suspension of nanoparticles, whereby the hydrophobic pharmaceutical agent becomes incorporated into the nanoparticles.

In some embodiments, the method includes the step of adding a polynucleotide to the aqueous suspension of nanoparticles, whereby the polynucleotide becomes non-covalently bound to the positively-charged polymers of the nanoparticles. In some embodiments, two or more polynucleotides are added to the aqueous suspension and become bound to the positively-charged polymers of the hypoxia-sensitive polynucleotide-binding molecule.

In one aspect, the invention is a method of treating a disease or condition associated with a hypoxic cell or tissue, the method including administering to a subject having or suspected of having the disease or condition a nanoparticle composition of the invention.

In some embodiments, the disease or condition associated with a hypoxic cell or tissue is cancer. In some embodiments, the cancer is associated with a solid tumor.

In some embodiments, the nanoparticle composition is administered by a parenteral route. In some embodiments, the parenteral administration route is intravascular administration, peri- and intra-tissue administration, subcutaneous injection or deposition, subcutaneous infusion, intraocular administration, or direct application at or near a site of neovascularization.

In some embodiments, the nanoparticle comprises a polynucleotide. In some embodiments, the polynucleotide targets the expression of one or more of survivin, Eg5, EGFR, XIAP, CDC45L, SUV420h1, WEE1, HDAC2, RBX 1, CDK4, CSN5, FOXM1, R1 (RAM2), LSD1, CSTF2, Nectin-4, ERCC6L, PKIB, NAALADL2, PRMT1, COPZ1, SYNGR4, P-glycoprotein, VEGFR, and VEGF.

In some embodiments, the nanoparticle comprises a hydrophobic pharmaceutical agent. In some embodiments, the hydrophobic pharmaceutical agent is one or more of altretamine, aminoglutethimide, amsacrine (m-AMSA), azacitidine, baccatin III, bleomycin, busulfan, carmustine (BCNU), chlorambucil, cytarabine HCl, dacarbazine, dactinomycin, daunorubicin, docetaxel, doxorubicin, etoposide (VP-16), 5-fluorouracil, floxuridine, flutamide, hydroxyurea, ifosfamide, leuprolide acetate, lomustine (CCNU), melphalan, methotrexate, mitomycin, mitotane (o.p'-DDD), octreotide, paclitaxel, pentostatin, plicamycin, procarbazine HCl, semustine (methyl-CCNU), streptozocin, tamoxifen citrate, teniposide (VM-26), thioguanine, thiotepa, vindesine, vinblastine, and vincristine sulfate.

In one aspect, the invention is a kit for use in treating a disease or condition associated with a hypoxic cell or tissue, the kit including a hypoxia-sensitive polynucleotide-binding molecule of the invention and packaging therefor.

In some embodiments, the hypoxia-sensitive polynucleotide-binding molecule is provided as a dry powder or film. In some embodiments, the hypoxia-sensitive polynucleotide-binding molecule is provided in the form of an aqueous suspension containing a plurality of nanoparticles containing the hypoxia-sensitive polynucleotide-binding molecules.

In some embodiments, the kit includes a polynucleotide.

In some embodiments, the kit includes a hydrophobic pharmaceutical agent.

In some embodiments, the kit includes instructions for reconstituting the hypoxia-sensitive polynucleotide-binding molecule as micelles in an aqueous suspension. In some embodiments, the kit includes instructions for forming a nanoparticle composition containing the hypoxia-sensitive polynucleotide-binding molecule and a polynucleotide. In some embodiments, the kit includes instructions for forming a nanoparticle composition containing the hypoxia-sensitive polynucleotide-binding molecule and a hydrophobic pharmaceutical agent. In some embodiments, the kit includes instructions for use of the kit for treating a disease or condition associated with a hypoxic cell or tissue according to a method of the invention. In some embodiments, the kit includes instructions for forming non-covalent bonds between the polynucleotide and the nanoparticle composition.

In one aspect, the invention is a kit for treating a disease or condition associated with a hypoxic cell or tissue, the kit including a nanoparticle composition containing the hypoxia-sensitive polynucleotide-binding molecule of the invention and packaging therefor.

In one aspect, the invention is a kit for treating a disease or condition associated with a hypoxic cell or tissue, the kit including a pharmaceutical composition containing the hypoxia-sensitive polynucleotide-binding molecule of the invention and packaging therefor.

### BRIEF DESCRIPTION OF THE DRAWINGS

FIG. 1 is a schematic illustration of a hypoxia-sensitive polynucleotide-binding molecule of the invention.

FIG. 2 is a proposed mechanism of siRNA internalization by PAPD polymers in hypoxic tumor microenvironment.

FIG. 3 shows the synthesis scheme of a molecule of the invention, PEG-Azo-PEI-DOPE. In reaction (i), polyethylene glycol 2000-N-hydroxysuccinamide ester is reacted with azobenzene-4,4′-dicarboxylic acid to create the PEG-Azo product. In reaction (ii), the PEG-Azo product is reacted with branched polyethylenimine, average molecular weight 1800 da, to create the PEG-Azo-PEI product. In reaction (iii), the PEG-Azo-PEI product is reacted with 1,2-dioleoyl-sn-glycero-3-phosphoethanolamine-N-(glutaryl) to create PEG-Azo-PEI-PE.

FIG. 4a is a graph of fluorescence from ethidium bromide in the presence of siRNA bound to polymers at various N/P ratios. Polymers tested were PEI 1.8 kDa (open diamonds), PAPD (Fopen circles), PEI (1.8 kDa) polyplexes treated with heparin (solid diamonds), PAPD treated with heparin (solid circles). FIG. 4b is an agarose gel of an RNAse protection assay. Samples were untreated free siRNA (lane 1), RNAse-treated free siRNA (lane 2), none (empty lane) (lane 3), PAPD polyplexes, N/P 40 (lane 4), RNAse-treated PAPD polyplexes, N/P 40, treated with RNAse and heparin (lane 5), PAPD polyplexes, N/P 60 (lane 6), PAPD polyplexes, N/P 60, treated with RNAse and heparin (lane 7), PEG-PEI-DOPE(PPD) polyplexes, N/P 40 (lane 8), PPD polyplexes, N/P 40, treated with RNAse and heparin (lane 9), PPD polyplexes, N/P 60 (lane 10), PPD polyplexes, N/P 60, treated with RNAse and heparin (lane 11), and none (empty lane) (lane 12). FIG. 4c is a graph showing siRNA signal from PAPD polyplexes prepared at an N/P ratio of 40 and incubated 2 h in PBS (1), 2.0% FBS media (2), 10% FBS N₂-bubbled media (3), 10% FBS N₂-bubbled media and microsomes (4), PBS followed by heparin treatment (5). * indicates p<0.05 and ** indicates p<0.01 compared with PBS-treated sample. FIG. 4d is a transmission electron microscopy micrograph of PAPD polyplexes in PBS showing a rodlike structure; scale bar represents 100 nm. FIG. 4e is a graph showing the zeta potential of PAPD/siRNA complexes prepared at an N/P of 40 after incubation with PBS. FIG. 4f is a graph showing the zeta potential of PAPD/siRNA complexes prepared at an N/P of 40 after incubation with N₂-bubbled PBS containing microsomes (f).

FIG. 5a shows representative histogram plots of internalized siRNA by cells cultured in a monolayer under hypoxia in the presence of 10% FBS. Cells were treated with PBS (1), free FAM-siRNA (2), PEG-PEI-DOPE/siRNA complexes (3), and PEG-Azo-PEI-DOPE/siRNA complexes (4). FIG. 5b is graph of the geometric mean of fluorescence of A549 cells incubated 24 h with the same formulations as in FIG. 4B under normoxia (white bars) and hypoxia (black bars). FIG. 5c shows confocal microscopic images of NCI-ADRRES spheroids after incubation for 4 h under normoxia and hypoxia with DY 547-labeled siRNA. Scale bar represents 250 mm. FIG. 5d is a graph of DY 547 fluorescence from the surface of spheroids after incubation with free siRNA (open diamonds for normoxia, solid diamonds for hypoxia), PEG-Azo-PEI-DOPE/siRNA (open triangles for normoxia, solid triangles for hypoxia) and PEG-PEI-DOPE/siRNA (open circles for normoxia, solid circles for hypoxia). FIG. 5e is a graph of average intensity of fluorescence at 120 mm from surface of spheroids after treatment with PEG-Azo-PEIDOPE/siRNA (PAPD) and PEG-PEI-DOPE/siRNA (PPD) under hypoxia. * indicates p<0.05 compared with PAPD/DY 547 siRNA complexes.

FIG. 6a is a graph of relative geometric mean fluorescence from FACS analysis of HeLa/GFP cells transfected with PEG-Azo-PEI-DOPE (PAPD)/siRNA complexes in the presence of 10% FBS under normoxic (NX) or hypoxic (HX) conditions. Polyplexes were prepared at N/P ratios of 40 and 60 with anti-GFP siRNA (black bars) or scrambled siRNA (white bars). Lipofectamine2000 (LFA) was used as a positive control. * indicates p<0.05 and ** indicates p<0.01 compared with scrambled siRNA complexes. FIG. 6b is a graph of relative geometric mean fluorescence from FACS analysis of HeLa/GFP cells transfected with PEG-PEI-DOPE (PPD)/siRNA complexes in the presence of 10% FBS. Polyplexes were prepared at N/P ratios of 40 and 60 with anti-GFP siRNA (black bars) or scrambled siRNA (white bars). Lipofectamine2000 (LFA) was used as a positive control. FIG. 6c shows confocal laser scanning microscopic images of HeLa/GFP cells transfected with Rhodamine B labeled copolymers PEG-Azo-Rhodamine-PEI-DOPE (PARPD), PEG-Rhodamine-PEI-DOPE (PRPD) and GFP siRNA under normoxia. FIG. 6d shows confocal laser scanning microscopic images of HeLa/GFP cells transfected with Rhodamine B labeled copolymers PEG-Azo-Rhodamine-PEI-DOPE (PARPD), PEG-Rhodamine-PEI-DOPE (PRPD) and GFP siRNA under hypoxia. FIG. 6e is graph of mean pixel intensities of GFP after transfection of HeLa/GFP cells under normoxia (white bars) and hypoxia (black bars) with PBS (1), free siRNA (2), PARPD (3), and PRPD (4). * indicates p<0.05 compared with normoxia. FIG. 6f is graph of mean pixel intensities of Rhodamine B after transfection of HeLa/GFP cells under normoxia (white bars) and hypoxia (black bars) with PBS (1), free siRNA (2), PARPD (3), and PRPD (4). ** indicates p<0.01 compared with normoxia.

FIG. 7 is graph showing relative GFP expression after transfection of NCI-ADR-RES/GFP and A2780/GFP cells under normoxia and hypoxia. Cells were transfected with free siRNA, PEG-Azo-PEI-DOPE/siRNA (PAPD), PEG-PEI-DOPE/siRNA (PPD), or Lipofectamine/siRNA (LFA) and analyzed after 48 hours. PAPD and PPD complexes were prepared at an N/P ratio of 60. Both anti-GFP siRNA (black bars) and scrambled siRNA (white bars) were used. * indicates p<0.05 and ** indicates p<0.01 compared with complexes formed with scrambled siRNA.

FIG. 8 is a graph of relative geometric mean fluorescence of cells from tumors from mice treated either PBS, PRPD, or PARPD. * indicates p<0.05 compared with PBS-treated or PRPD-treated mice.

FIG. 9a shows ex vivo fluorescence optical imaging of tumors 48 h after intravenous injection of PBS (n=4), PEG-Azo-PEI-DOPE/anti-GFP siRNA complexes prepared with (PAPD/siGFP, n=4), and PEG-Azo-PEI-DOPE/negative siRNA complexes (PAPD/siNeg, n=3) at a dose of 1.5 mgkg⁻¹ of siRNA in 200 mL PBS. FIG. 9b is a graph of relative GFP fluorescence from tumors. Student's t test was performed. * indicates p<0.05 compared to PBS or PAPD/siNeg. FIG. 9c shows the results of flow cytmetric analysis of dissociated tumors from mice after injection with PBS (dashed line), PAPD/siGFP (dot-dashed line), and PAPD/siNeg (solid line). FIG. 9d is a representative histogram of cell-associated GFP fluorescence from cells analyzed in FIG. 9c. Only PAPD/siGFP led to a significant decrease of GFP expression by student's t test. ** indicates p<0.01 compared to PBS-treated sample, # indicates p<0.001 compared to PAPD/siNeg-treated sample.

### DETAILED DESCRIPTION OF THE INVENTION

The present invention provides compositions and methods for the delivery of a polynucleotide, hydrophobic pharmaceutical agent, or both to a hypoxic cell or tissue. The compositions and methods employ an amphipathic molecule that self-assembles into micellar nanoparticles. The micellar nanocarrier possesses several key features for delivery of polynucleotides and hydrophobic drugs, including (i) excellent stability; (ii) efficient condensation of polynucleotides by a positively-charged polymer; (iii) hydrophobic drug solubilization in the lipid “core”; (iv) passive tumor targeting via the enhanced permeability and retention (EPR) effect; (v) tumor targeting triggered by the hypoxia-sensitive moiety; and (vi) enhanced cell internalization after hypoxia-dependent exposure of the previously hidden positively-charged polymer. These cooperative functions ensure the improved tumor targetability, enhanced tumor cell internalization, and synergistic antitumor activity of co-loaded siRNA and drug.

As used herein, “hydrophobic” refers to a molecule or portion of a molecule that has greater solubility in an organic solvent than in an aqueous medium, and “hydrophilic” refers to a molecule or portion of a molecule that has greater solubility in an aqueous medium than in an organic solvent. One way of assessing hydrophobicity/hydrophilicity is to determine the partition coefficient of a molecule at room temperature (20-25° C.) between octanol and water, as reflected in the log P_{OW}. A molecule with a log P_{OW} above a threshold value is considered hydrophobic, and a molecule with a log P_{OW} below a threshold is considered hydrophilic.

As used herein, the term “uncharged” refers to a molecule or portion of a molecule that is not ionic in an aqueous medium at physiological pH and temperature, the term “positively-charged” refers to a molecule or portion of a molecule that is cationic at physiological pH and temperature, and the term “negatively-charged” refers to a molecule or portion of a molecule that is anionic at physiological pH and temperature.

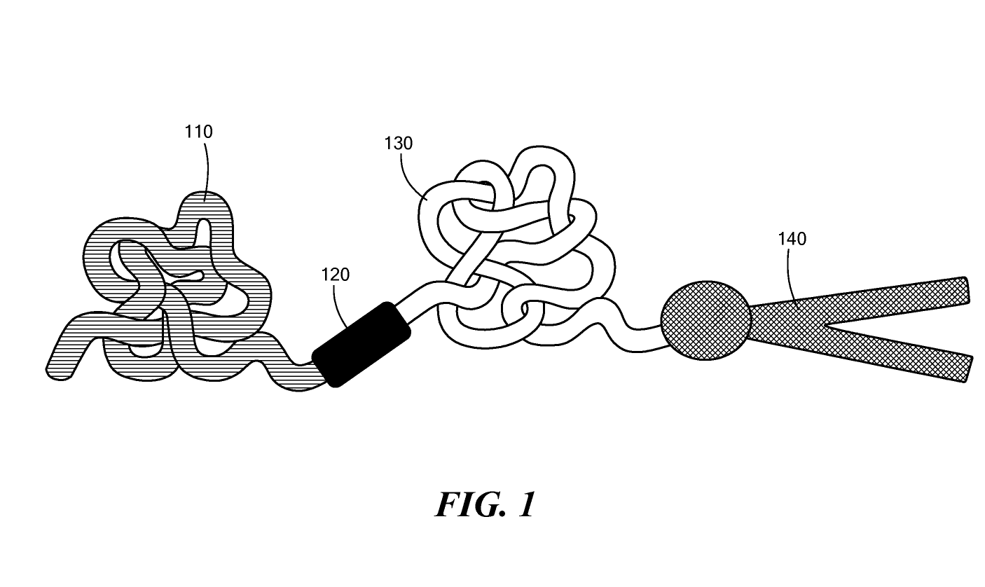

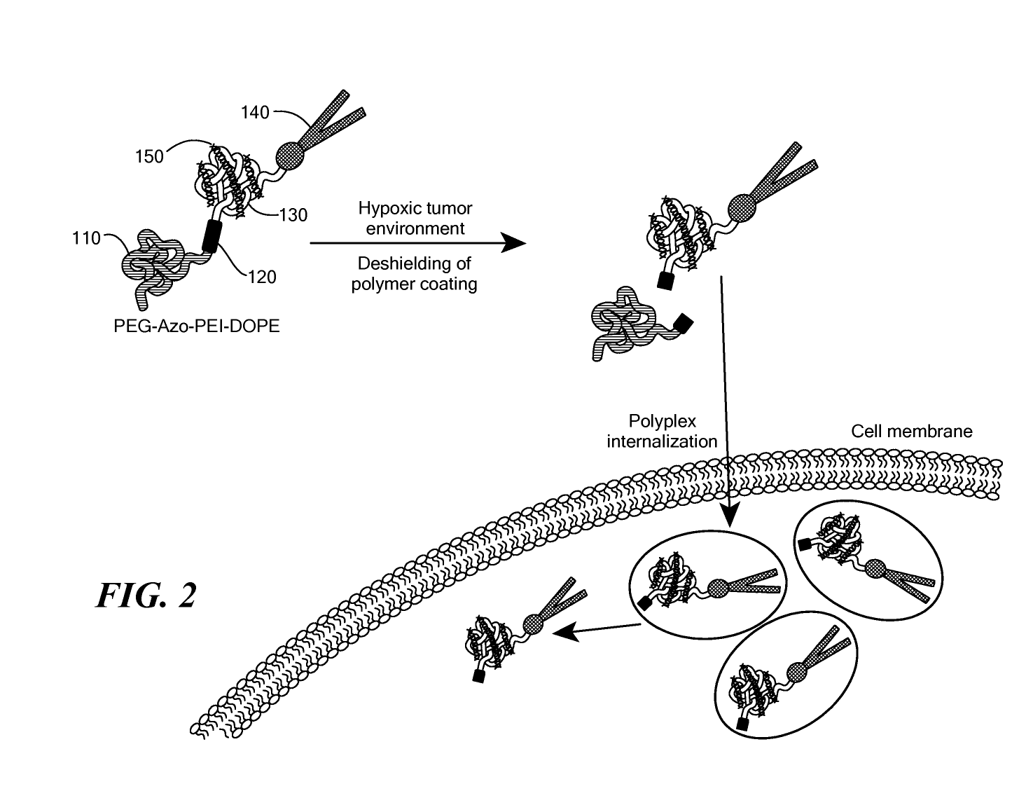

The invention includes a hypoxia-sensitive, polynucleotide-binding molecule that can form micellar nanoparticles. As shown in FIG. 1, the molecule contains a series of covalent linkages between an uncharged hydrophilic polymer (110), a hypoxia-sensitive moiety (120), a positively-charged polymer (130), and an amphipathic molecule such as a phospholipid (140). Each part of the molecule serves a different function. Intermolecular interactions between the fatty acid chains of the phospholipid promote assembly of the molecules into a micellar nanoparticle with a hydrophobic core, in which a hydrophobic pharmaceutical agent can be stably solubilized. As shown in FIG. 2, the positively-charged polymer electrostatically interacts with the negatively-charged phosphate backbone of a polynucleotide (150) to promote condensation of the polynucleotide. This condensation protects the polynucleotide from nucleases and thus renders it stable for in vivo delivery. The uncharged hydrophilic polymer forms the surface of the nanoparticle in an aqueous environment and shields the positively-charged polymer from other solutes. Highly charged nanoparticles are cleared from the circulation more rapidly, so the charge shielding provided by the uncharged polymer extends the blood circulation time of the nanoparticle. However, the charge shielding also impairs cellular uptake of nanoparticles and the cargo that they carry. This side effect is overcome by the hypoxia-sensitive moiety, which contains a covalent bond that can cleaved in a hypoxic and reducing environment. Cleavage of the hypoxia-sensitive moiety results in the de-shielding of the nanoparticle and exposure of the positively-charged polymer, which facilitates cellular uptake of the nanoparticle. Consequently, the nanoparticle of the invention can preferentially deliver polynucleotides and/or hydrophobic pharmaceutical agents to a hypoxic cell or tissue.

The hypoxia-sensitive moiety may be any molecule that has a covalent bond that can be cleaved in a reducing environment. For example and without limitation, it may be azobenzene or a derivative thereof or a nitroimidazole derivative. The azobenzene derivative may be an azobenzene dicarboxamide with one carboxamide substituent on each aromatic ring. For example, the azobenzene dicarboxamide substituent may be azobenzene-4,4′-dicarboxamide. However, any arrangement of the carboxamide substituents on the aromatic rings is possible. For example, the azobenzene derivative may have a carboxamide substituent at the 2, 3, 4, 5, or 6 position of the first aromatic ring and at the 2′, 3′, 4′, 5′, or 6′ position of the second aromatic ring. The azobenzene derivative may be symmetric or asymmetric. The azobenzene derivative may have other types substituents, for example, an alkyl group, ester group, or any other stable substituent.

The uncharged hydrophilic polymer may be any water-soluble polymer that is uncharged at physiological pH and temperature and has a flexible main chain. For example and without limitation, the uncharged hydrophilic polymer may be polyethylene glycol, polyvinylpyrrolidone, or polyacrylamide. If the uncharged hydrophilic polymer is polyethylene glycol, it may have an average molecular weight from about 1000 to about 10,000 daltons, from about 1000 to about 5000 daltons, from about 2000 to about 4000 daltons, or about 2000 daltons. The uncharged hydrophilic polymer may be a derivative of molecule described above. For example and without limitation, the uncharged hydrophilic polymer may be polyethylene glycol N-hydroxysuccinamide ester, or it may be another derivatized form of polyethylene glycol.

The positively charged polymer may be any polymer that is positively charged at physiological pH and temperature. For example and without limitation, the positively-charged polymer may be polyethylenimine, polylysine, a cationic peptide, poly(dl-lactide-co-glycolide), poly(amidoamine), or poly(propylenimine). If the positively-charged polymer is polyethylenimine, it may have an average molecular weight from about 500 daltons to about 5000 daltons, from about 1000 to about 2000 daltons, from about 5000 to about 20,000 daltons, from about 20,000 to about 30,000 daltons, about 1800 daltons, or about 25,000 daltons. The polyethylenimine may have a linear structure, a branched structure, or a dendrimeric structure. The positively-charged polymer may be a derivative of molecule described above.

The phospholipid may be any stable phospholipid with amphipathic properties or another type of amphipathic molecule. For example and without limitation, the phospholipid may be phosphatidic acid, phosphatidylethanolamine, phosphatidylcholine, phosphatidylserine, phosphatidylinositol, phosphotidylglycerol, or a sphingolipid. The fatty acid chains in the phospholipid may be any length or structure that is compatible that allows the hypoxia-sensitive, polynucleotide-binding molecule to form micelles. For example, the fatty acid chains may have from 9 to 20 carbon atoms, from 10 to 20 carbon atoms, from 12 to 20 carbon atoms, from 14 to 20 carbon atoms, or from 16 to 20 carbon atoms. The fatty acid chains in the phospholipid may be saturated, monounsaturated, diunsaturated, or triunsaturated. The unsaturated fatty acid side chains may have carbon-carbon double bonds in either a cis or trans configuration.

The covalent linkage may be any covalent bonds that is stable at physiological pH and temperature. For example and without limitation, the covalent linkage may be a peptide bond, amide bond, ester bond, ether bond, alkyl bond, carbonyl bond, alkenyl bond, thioether bond, disulfide bond, or azide bond. The covalent linkage may be cyclical. For example and without limitation, the covalent linkage may be a 1,2,3-triazole or cyclohexene.

The micellar nanoparticles may assume various sizes and morphologies. For example and without limitation, they may be spherical or worm-like (long, essentially cylindrical, and flexible). The micellar nanoparticles may have an average diameter from about 10 nm to about 100 nm, from about 10 nm to about 100 nm, from about 10 nm to about 50 nm, or from about 20 to about 40 nm. The micellar nanoparticles may consist only of the hypoxia-sensitive polynucleotide-binding molecule described herein.

Alternatively, the micellar nanoparticles may contain one or more polynucleotides non-covalently bound to the positively charged polymer of the hypoxia-sensitive, polynucleotide-binding molecule. The polynucleotide may be any nucleic acid molecule. For example, the polynucleotide may be a molecule of single-stranded RNA, double-stranded RNA, single-stranded DNA, or double-stranded RNA. The polynucleotide may be a molecule of siRNA. The polynucleotide may be an oligonucleotide. For example, the polynucleotide may be an antisense oligonucleotide. The polynucleotide may target a gene involved in cancer. For example, the polynucleotide may target survivin, Eg5, EGFR, XIAP, CDC45L, SUV420h1, WEE1, HDAC2, RBX 1, CDK4, CSN5, FOXM1, R1 (RAM2), LSD1, CSTF2, Nectin-4, ERCC6L, PKIB, NAALADL2, PRMT1, COPZ1, SYNGR4, P-glycoprotein, VEGFR, and/or VEGF. The micellar nanoparticles may have two or more different species of polynucleotides.

The micellar nanoparticles may be formed by adding the hypoxia-sensitive, polynucleotide-binding molecule and the polynucleotide in a ratio that promotes condensation of the polynucleotide in the nanoparticle. For example, a micellar nanoparticle made by adding a hypoxia-sensitive, polynucleotide-binding molecule having polyethylenimine as its positively-charged polymer and the polynucleotide in a nitrogen:phosphate ratio of about 1:1 to about 1:50, about 1:2 to about 1:50, about 1:5 to about 1:50, about 1:5 to about 1:25, about 1:10 to about 1:25. The degree of condensation may be assess by change in diameter of nanoparticle size, by protection of the polynucleotide from nuclease digestion, or by other methods.

The micellar nanoparticles may contain one or more hydrophobic pharmaceutical agents. The hydrophobic pharmaceutical agent may be any hydrophobic compound that can be used to treat a disease or condition. For example, the hydrophobic pharmaceutical agent may be an anti-cancer agent. For example, the hydrophobic pharmaceutical agent may be altretamine, aminoglutethimide, amsacrine (m-AMSA), azacitidine, baccatin III, bleomycin, busulfan, carmustine (BCNU), chlorambucil, cytarabine HCl, dacarbazine, dactinomycin, daunorubicin, docetaxel, doxorubicin, etoposide (VP-16), 5-fluorouracil, floxuridine, flutamide, hydroxyurea, ifosfamide, leuprolide acetate, lomustine (CCNU), melphalan, methotrexate, mitomycin, mitotane (o.p′-DDD), octreotide, paclitaxel, pentostatin, plicamycin, procarbazine HCl, semustine (methyl-CCNU), streptozocin, tamoxifen citrate, teniposide (VM-26), thioguanine, thiotepa, vindesine, vinblastine, vincristine sulfate, or any combination thereof. The hydrophobic pharmaceutical agent may be a small molecule drug having a molecular weight of less than 2000 daltons, less than 1500 daltons, less than 1000 daltons, or less than 500 daltons. The hydrophobicity is such that the pharmaceutical agent is soluble in the hydrophobic core of a micellar nanoparticle of the invention.

The hypoxic cell or tissue may be associated with a disease or condition. For example, the hypoxic cell or tissue may be associated with cancer. The cancer may be associated with a solid tumor. For example, the cancer may be uterine cancer, cervical cancer, prostate cancer, ovarian cancer, sarcoma, or head and neck cancer.

The hypoxia-sensitive moiety within the micellar nanoparticle is cleavable in a hypoxic environment. The hypoxia-sensitive moiety covalently links the uncharged polymer to the rest of the hypoxia-sensitive, polynucleotide-binding molecule. Consequently, cleavage of the hypoxia-sensitive moiety in a hypoxic environment results in release of the uncharged hydrophilic polymers from the nanoparticles. The uncharged hydrophilic polymers shield the charge of the nanoparticle from the aqueous environment, and hypoxia-dependent cleavage of the molecule causes the charge of the nanoparticle to become deshielded. The deshielding of the nanoparticle's charge promotes cellular uptake of the nanoparticle (FIG. 2). Thus, when the nanoparticle contains one more bound polynucleotides and hydrophobic pharmaceutical agents, cleavage of the hypoxia-sensitive moiety increases the cellular uptake of these components as well. In addition, the hypoxia-dependent deshielding of the nanoparticle facilitates release of the polynucleotide(s) and/or hydrophobic pharmaceutical agent(s) from an intracellular vesicular compartment into the cytoplasm (FIG. 2).

The micellar nanoparticle may be suspended in an aqueous medium for use or storage. The aqueous medium may contain excipients to promote the stability of the nanoparticles or their effectiveness in delivery of polynucleotides and/or hydrophobic pharmaceutical agents. Such excipients are well known in the art. For example and without limitation, the suspension of micellar nanoparticles may contain one or more buffers, electrolytes, agents to prevent aggregation of nanoparticles, agents to prevent adherence of nanoparticles to the surfaces of containers, cryoprotectants, and/or pH indicators.

The invention includes methods of making the hypoxia-sensitive, polynucleotide-binding molecules of the invention from the individual chemical components. One step of the method entails reacting a reactive group on the uncharged hydrophilic polymer with a reactive group on the hypoxia-sensitive moiety to form a covalent linkage between these two components. In another step, a reactive group on the hypoxia-sensitive moiety is reacted with a reactive group on the positively-charged polymer to form a covalent linkage between these two components. In another step, a reactive group on the positively-charged polymer is reacted with a reactive group on the phospholipid to form a covalent linkage between these two components.

The steps required to make the hypoxia-sensitive, polynucleotide-binding molecules of the invention can be performed in any order. For example, the uncharged hydrophilic polymer and hypoxia-sensitive moiety can be joined first, the hypoxia-sensitive moiety and positively-charged polymer can be joined second, and the positively-charged polymer and phospholipid can be joined third. Alternatively, the uncharged hydrophilic polymer and hypoxia-sensitive moiety can be joined first, and the positively-charged polymer and phospholipid can be joined second, and the hypoxia-sensitive moiety and positively-charged polymer can be joined third. Alternatively, the hypoxia-sensitive moiety and positively-charged polymer can be joined first, the uncharged hydrophilic polymer and hypoxia-sensitive moiety can be joined second, and the positively-charged polymer and phospholipid can be joined third. Alternatively, the hypoxia-sensitive moiety and positively-charged polymer can be joined first, the positively-charged polymer and phospholipid can be joined second, and the uncharged hydrophilic polymer and hypoxia-sensitive moiety can be joined third. Alternatively, the positively-charged polymer and phospholipid can be joined first, the uncharged hydrophilic polymer and hypoxia-sensitive moiety can be joined second, and the hypoxia-sensitive moiety and positively-charged polymer can be joined third. Alternatively, the positively-charged polymer and phospholipid can be joined first, the hypoxia-sensitive moiety and positively-charged polymer can be joined second, and the uncharged hydrophilic polymer and hypoxia-sensitive moiety can be joined third. It will be understood by one of ordinary skill in the art that particular starting reactants of the reaction in each step of the method will vary depending on the sequence in which the steps are performed. Therefore, the starting reagents may be the individual components described above, or they may composite molecules consisting of two or three of the individual components described above that have been covalently linked according to the manner required by an earlier step of the method.

The individual steps of the method are performed to give products that have each of the starting reactants combined in a 1:1 molar ratio. The starting reactants may be present in a 1:1 molar ratio or in unequal molar amounts. Chemical reactions may be performed in organic solvents or in aqueous media. In addition to the reactants and solvents, the reactions may contain additional components as catalysts, solubilizers, and the like. For example, and without limitation, the reactions may include N-(3-dimethylaminopropyl)N′-ethylcarbodiimide hydrochloride, N-hydroxysuccinimide, pyridine, 4-dimethylaminopyridine, and/or triethylamine. Each step of the method may be performed in a single step or in a series of sub-steps. A sub-step may entail a chemical reaction, an analytical method, a purification method, an exchange of solvent or medium, or any other process necessary to complete a step of the method.

The reactants react via reactive groups. The reactive groups allow formation of specific covalent linkages between two reactants. The reactive groups may be inherent in the starting components, the reactive groups may be added by derivatizing the starting components prior to performing the reaction in which the desired covalent linkage is formed. A reactant may have a single reactive group of a particular species, which directs formation of particular covalent linkage to a specific site within the reactant. Therefore, the hypoxia-sensitive, polynucleotide-binding molecules of the invention can be made with one or more of the components having a specific orientation within the molecule. Alternatively, a reactant may have multiple reactive groups of a particular species, which allows formation of particular covalent linkage at multiple sites within the reactant. A reactant may have multiple species of reactive groups, thereby allowing formation of multiple different types of covalent linkages at distinct sites within the reactant. Therefore, the hypoxia-sensitive, polynucleotide-binding molecules of the invention can be made with one or more of the components having a varied orientation within the molecule. For example, and without limitation, the reactive group may be a thiol, dithiol, trithiol, acyl, amine, carboxylic acid, azide, alkene, maleimide, alcohol, alkyne, dienyl, phenol, ester, or N-glutaryl. The reactive group may be joined to the reactant via a linker, for example, an oligoethylene glycol chain.

The invention includes methods of making micellar nanoparticles containing the hypoxia-sensitive, polynucleotide-binding molecules of the invention. The method entails providing a solution of the hypoxia-sensitive, polynucleotide-binding molecule in an organic solvent and replacing the non-aqueous solvent with an aqueous medium to form an aqueous suspension comprising nanoparticles made up of the molecule. The organic solvent may be replaced by an aqueous medium by any method known in the art. For example, the organic solution of the hypoxia-sensitive, polynucleotide-binding molecule may be dialyzed against an aqueous medium to remove the organic solvent. Alternatively, the organic solvent may be evaporated to form a dry film of the hypoxia-sensitive, polynucleotide-binding molecule, which is then resuspended in an aqueous medium.

The methods of making micellar nanoparticles containing the hypoxia-sensitive, polynucleotide-binding molecules of the invention may include addition of other components. For example, a hydrophobic pharmaceutical agent may be included. One or more hydrophobic pharmaceutical agent mays be added to the organic solution containing the hypoxia-sensitive, polynucleotide-binding molecule, resulting in formation of micellar nanoparticles that contain the hydrophobic pharmaceutical agent(s). Alternatively, one or more hydrophobic pharmaceutical agents may be added to the aqueous suspension of micellar nanoparticles so that the hydrophobic pharmaceutical agent(s) is incorporated into the hydrophobic core of the nanoparticles. In another example, one or more polynucleotide(s) may be added to the aqueous suspension of micellar nanoparticles so that the polynucleotide(s) becomes non-covalently bound to the positively-charged polymer of the nanoparticle.

The invention includes methods of treating a disease or condition associated with a hypoxic cell or tissue by administering a composition of the micellar nanoparticles of the invention to a subject having or suspected of having the disease or condition. The nanoparticle composition may be administered by a parenteral route. For example, the nanoparticle composition may be administered by intravascular administration, peri- and intra-tissue administration, subcutaneous injection or deposition, subcutaneous infusion, intraocular administration, and direct application at or near a site of neovascularization.

The invention also includes kits for use in treating a disease or condition associated with a hypoxic cell or tissue. The kits may include a hypoxia-sensitive, polynucleotide-binding molecule of the invention. The hypoxia-sensitive, polynucleotide-binding molecule may be provided as a powder or dry film. The kit may include instructions for reconstituting the powder or dry film of hypoxia-sensitive, polynucleotide-binding molecule as micellar nanoparticles in an aqueous suspension. Alternatively, the hypoxia-sensitive, polynucleotide-binding molecule may be provided as micellar nanoparticles in an aqueous suspension.

The kit may include micellar nanoparticles of the invention. The micellar nanoparticles may consist only of the hypoxia-sensitive, polynucleotide-binding molecule of the invention. Alternatively, the micellar nanoparticles may also include other components. For example, the micellar nanoparticles may also include a polynucleotide and/or a hydrophobic pharmaceutical agent.

The kit may include a pharmaceutical composition of the invention that includes a suspension of micellar nanoparticles containing a hypoxia-sensitive, polynucleotide-binding molecule.

The kit may also include other components in separate containers. For example, the kit may include a polynucleotide and/or a hydrophobic pharmaceutical agent.

The kit may also include instructions for preparing and using the compositions of the invention. For example, the kit may include instructions for forming a nanoparticle composition containing the hypoxia-sensitive, polynucleotide-binding molecule of the invention and a polynucleotide and/or hydrophobic pharmaceutical agent. The kit may include instructions for forming non-covalent bonds between a polynucleotide and a micellar nanoparticle of the invention. The kit may include instruction for incorporating a hydrophobic pharmaceutical agent into a micellar nanoparticle of the invention. The kit may include instructions for use of the kit in treating a disease or condition associated with a hypoxic cell or tissue according to a method of the invention.

### EXAMPLES

### Example 1

### Materials and Methods

Materials.

The sequence of anti-GFP siRNA was 5′-AUGAACUUCAGGGUCAGCUdTdT-3′ (sense) (SEQ ID NO:1). [18] Pimonidazole hydrochloride and mouse antibody against reduced pimonidazole adducts were from Hydroxyprobe, Inc. (Burlington, Mass.). Goat anti-mouse PE (phycoerythrin)-conjugated anti-mouse antibody and Mini Collect heparin-coated tubes were from Santa Cruz Biotechnology (Santa Cruz, Calif.). Goat anti-mouse TRITC-conjugated antibody and rat liver microsomes were from Invitrogen (Grand Island, N.Y.). Mouse myeloma ascites IgG2a was purchased from MP Biomedicals (Santa Ana, Calif.). pEGFP-N1 plasmid encoding EGFP (enhanced green fluorescent protein) was from Erlim Biopharmaceuticals (Hayward, Calif.).

A2780/GFP and NCI-ADR-RES/GFP Cells.

A2780 cells stably expressing GFP (A2780/GFP) and NCI-ADR-RES cells stably expressing GFP (NCI-ADRRES/GFP) were obtained by antibiotic selection using 500 μg/mL G418 as in [19] after transfection of A2780 or NCIADR-RES cells with pEGFP-N1 pDNA complexed with Lipofectamine followed by screening of GFP positive clones by flow cytometry. GFP clones consisted of >90% of GFP positive cells (data not shown). GFP expression by A2780/GFP and NCI-ADR-RES/GFP cells relative to parent A2780, NCI-ADR-RES/GFP cells was analyzed by flow cytometry. Cells were seeded in 24-well plates at a density of 1.4×10⁵ cells/well. The next day, they were detached for flow cytometry analysis. More than 90% were GFP-positive cells for both A2780/GFP and NCI-ADR-RES/GFP cells. (10,000 events were recorded).

Synthesis and Characterization Procedure for PEG-Azo-PEI-DOPE and PEG-PEI-DOPE.

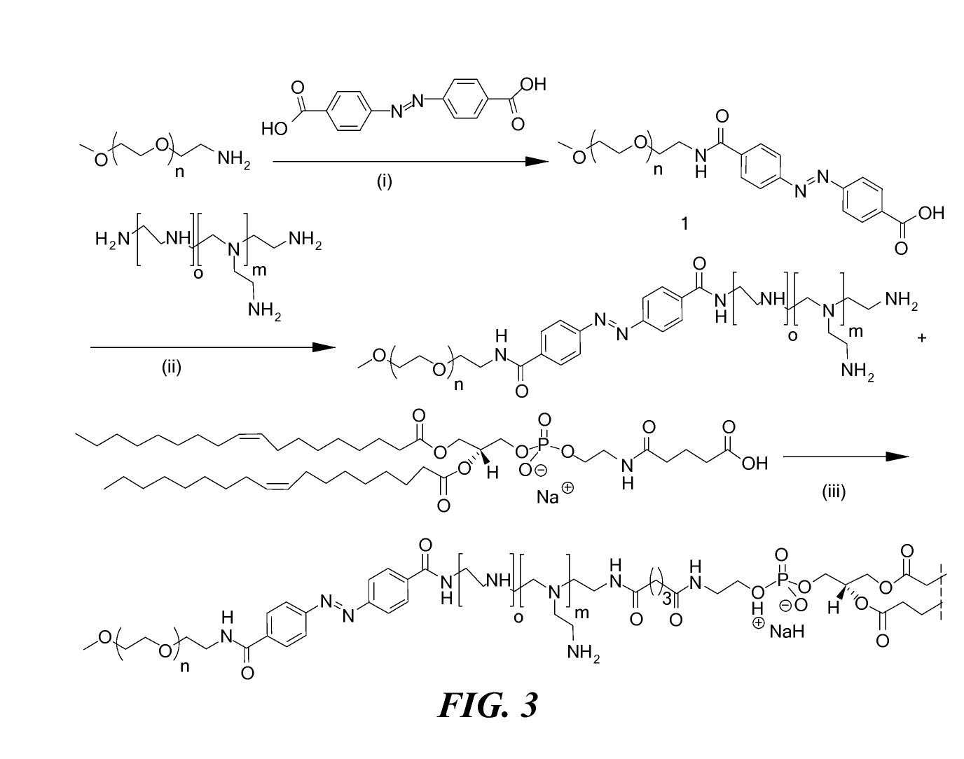

The procedures used are summarized in FIG. 3. To obtain PEG-Azo-PEI-DOPE, an azobenzene linker was introduced between PEG and PEI-DOPE. The mPEG-Amine was reacted with Azobenzene-4,4′-dicarboxylic acid. The acid-amine coupling reaction was performed in the presence of excess dicarboxylic acid. The acid group activating reagents, EDC and NHS were used in same equivalents to minimize the activation of the two acid groups in Azobenzene-4,4′-dicarboxylic acid. The poor solubility of Azobenzene-4,4′-dicarboxylic acid in CHCl₃ was overcome using pyridine. The ¹H-NMR spectra (data not shown) of PEG-Azo-Acid (1) showed the characteristic multiplet signal of the protons from PEG at δ 3.58-3.70. Peaks at δ 7.93-8.01 (m, Ar—H—N═N—), 8.19-8.21 (d, Ar—H—CONH) and the multiplet at δ 2.53-2.71 revealed the presence of Azo and PEI, respectively. Conjugation of DOPE in polymer 3 was confirmed by the appearance of characteristic signals from DOPE at regions δ 0.85-0.88 from the two terminal methyl groups, δ 1.25-1.28 from the protons of alkyl chains, and two other peaks in the region of δ 5.19-5.33 from the double bond in the alkyl chain. PEG-Azo-PEI-DOPE showed characteristic signals from PEG, Azobenzene, PEI and DOPE. The polymers with UV absorption (1-3) showed retention times of 0.91-0.94 in UV-TIC spectra with maximum UV absorbance at. 328.6-330.6 nm, obtained from analytical LC-MS (data not shown).

Singlet, doublet, triplet, multiplet and broad signals in NMR are denoted by s, d, t, m, and b, respectively. Obtention of PEG-Azo-PEI-PE was evidenced by ¹H NMR spectroscopy with characteristic peaks of PEG-Azo-Acid (1): δ 1.11 (t, H, —O—CH₃), 1.24-1.26 (m), 1.9-2.0 (sharp m), 2.7-3.7 (m), 7.93-8.01 (m, Ar—H—N═N—), 8.19-8.21 (d, Ar—H—CONH). Conjugation through an Azo linker was confirmed by LC-MS (retention time in UV-TC spectra. 0.94 min) with a characteristic UV absorbance at 328.6. nm. For PEG-Azo-PEI (2): δ 1.19 (m), 2.2 (sharp m), 2.53-2.71 (m), 3.31-3.69 (m), 4.05-4.2 (m), 7.8-8.0 (m, Ar—H). Conjugation through an Azo linker was again confirmed by LC-MS (retention time in UV-TC spectra. 0.95 min) with a characteristic UV absorbance at 330.6 nm. [20] Similar peaks were detected for PEG-Azo-PEI-DOPE (3): δ 0.85-0.88 (t, 6H, (—CH₂CH₃)₂), 1.25-1.28 (m), 1.97-2.00 (sharp m), 2.22-2.27 (m), 2.55 (bs), 3.53-3.54 (m), 3.56-4.5 (m), 5.19 (bs), 5.31-5.33 (sm), 7.98-8.00 (m, Ar—H). The retention time in UV-TC spectra was 0.91 min, with absorbance at 329.6 nm. The characteristic peaks of each component of PEG-PEI and PEG-PEIDOPE were detected by ¹H NMR as follows: ¹H-NMR of PEG-PEI (4): δ 2.52-2.81 (m), 3.36-3.97 (m). ¹H-NMR of PEG-PEI-DOPE (5): δ ¹H-NMR of PEG-PEI-DOPE (5): δ 0.84-0.87 (t, 6H, (—CH₂CH₃)₂), 1.22-1.27 (m), 1.96-1.99 (sharp m), 2.26-2.28 (m), 2.56 (bs), 3.25-3.68 (m), 3.80-4.4 (m), 5.19 (bs), 5.31-5.33 (sm). The reactions of PEG derivative with PEI and the PEGylated PEI with DOPE were performed using 1:1 molar ratio. Therefore, the molar ratio of PEG, PEI, and DOPE was 1:1:1. The integration values of specific peaks from the polymer blocks in the NMR data was consistent with the reagent ratio used for the reaction. Note that no detectable signal at 330 nm corresponding to Azo was detected from PEG-PEI-DOPE.

Procedure for the Syntheses of Rhodamine-Labeled PEI and Rhodamine-Labeled Polymers.

To a solution of PEI (200 mg, 111 μM) and triethylamine (30 μL) in CHCl₃, rhodamine B isothiocyanate (59.6 mg, 111 μM) dissolved in DMF/CHCl₃ (1:1) (500 μL) was added. The reaction mixture was stirred overnight under a nitrogen atmosphere at room temperature. The following day, the organic solvent was removed by rotary evaporation from the reaction mixture. The crude reaction mixture was dissolved in water and dialyzed using a cellulose ester membrane (MWCO, 1.0 KDa) against water for 1 day. The dialysate was freeze dried. The ¹H-NMR of Rh-PEI was as follows: δ 0.78-1.62 (m), 2.53-2.71 (m), 3.21-3.35 (m), 6.11-6.41 (m), 6.66-6.68 (d), 7.03-7.05 (d), 7.72-7.74 (d), 8.01-8.04 (m). The rhodamine-labeled polymers were synthesized using rhodamine-labeled PEI and characterized by ¹H NMR and LCMS (data not shown). The characteristic proton signal of rhodamine was observed in the ¹H-spectra of rhodamine-labeled polymers at δ 0.75-1.68 and δ 6.11-8.08 (data not shown). Azobenzene-containing polymers, PEG-Azo-RPEI-DOPE showed dual maximum absorbance emanated from the presence of the azobenzene linker and rhodamine (λₘₐₓ. 327.6 and 557.6 nm, respectively; data not shown). The ¹H-NMR of PEG-Azo-Rh-PEI was as follows: δ 0.82-0.93 (m), 1.06-1.31 (m), 2.13-2.23 (m), 2.60 (bs), 3.55-3.67 (m), 6.13-6.4 (m), 7.50-7.52 (d), 7.73-7.75 (d), 7.87-8.13 (m). The ¹H-NMR of PEG-Azo-Rh-PEI-DOPE was as follows: δ 0.87-0.88 (m), 1.25-1.31 (m), 1.98-2.00 (d), 2.13-2.25 (m), 2.50-3.70 (m), 3.80-4.40 (m), 5.20-5.32 (m), 6.13-6.40 (m), 7.00 (s), 7.35 (bs), 7.50-7.52 (d), 7.73-7.75 (d), 7.87-8.13 (m). The ¹H-NMR of PEG-Rh-PEI-DOPE was as follows: δ 0.82-0.89 (m), 1.26-1.29 (m), 1.98-2.01 (d), 2.27 (bs), 2.40-3.2 (m), 3.33-4.40 (m), 5.20-5.32 (m), 6.13-6.40 (m), 7.00 (s), 7.51-7.52 (d).

Microsome Stability Assay.

siRNA decondensation was determined using EtBr after incubation for various periods in DMEM media containing 10% FBS with and without 0.5 mg/mL rat liver microsomes and 50 μM NADPH as electron donor as in [21] in normoxic or hypoxic conditions. Hypoxia was generated by bubbling 100% nitrogen gas in line with. [22]

Aniline Release.

The release of aniline to assess cleavage of the azobenzene linker was evaluated as reported. [23] PEG-Azo-PEI-DOPE (20 μM) was incubated 2 h in DMEM media containing 10% FBS in normoxic and hypoxic conditions with and without 0.5 mg/mL rat liver microsomes before recording the absorbance at 400 nm. Hypoxic conditions were created by bubbling nitrogen in the media.

Size and Zeta Potential.

Zeta potential of PEG-Azo-PEI-DOPE/siRNA complexes prepared at an N/P ratio of 40 were recorded after 2 h incubation in PBS pH 7.4, and in N₂-bubbled PBS containing 0.5 mg/mL rat liver microsomes. Zeta potential were recorded with an Ultrasensitive Zeta Potential Analyzer instrument (Brookhaven Instruments, Holtsville, N.Y.). Samples containing microsomes were 0.2 μm-filtered before analysis.

Transmission Electron Microscopy.

Morphologies of PEG-Azo-PEI-DOPE/siRNA and PEG-PEI-DOPE/siRNA complexes at an N/P ratio of 60 were analyzed by transmission electron microscopy (TEM) with a Jeol, JEM-1010 microscope (Jeol, Tokyo) at a 40,000× magnification (scale bar represents 200 nm). Both complexes showed a rod-like morphology, comparable with morphologies of other complexes between nucleic acids and PEGylated polyelectrolytes. [24]

Cellular Viability.

Cell viability after treatments was measured with a Cell Titer Blue Cell viability assay (Promega, Madison, Wis.) for free polymers and complexes. [18] A549 and A2780 cells were seeded in 96-well plates at 3.0×10³ cells/well. The next day cells were incubated with free polymers or complexes for 48 h before determination of cellular viability.

Detection of Pimonidazole Adducts to Confirm Hypoxic Conditions.

Incubation of cells under hypoxic atmosphere was confirmed by Hydroxyprobe staining. [25] For monolayer cultures, A549 cells were seeded in 24-well plates at a density of 1.2×10⁵ cells/well. The next day cells were incubated for 3 h at 37° C. in humidified cell culture incubators under either normoxic (21% O₂, 5% CO₂) or hypoxic (0.5% O₂, 5% CO₂, nitrogen balanced) atmospheres with 100 μM pimonidazole hydrochloride. Then, cells were washed with PBS, detached with trypsin, methanol-permeabilized and stained with antibody against reduced pimonidazole adducts or an isotype-matched mouse antibody as control at a 1/100 dilution in PBS, 1% BSA for 1 h at RT. This was followed by staining with a secondary IgG PE-conjugated antibody at a 1/100 dilution for 1 h at RT. Lastly, cells were analyzed by flow cytometry with a FACSCalibur flow cytometer (Beckton Dickinson, Franklin Lakes N.J.). The cells were gated upon acquisition using forward versus side scatter to exclude debris and dead cells; 10,000 gated events were recorded (λₑₓ 488 nm, λₑₘ 585/42 nm). For spheroids, NCI-ADR-RES spheroids were incubated 3 h under hypoxia with 100 μM pimonidazole hydrochloride. [26] Spheroids were then fixed with neutral-buffered formalin and cut in 15 μm sections. [27] Sections were probed with an anti-pimonidazole adducts antibody ( 1/100 in PBS, 1% BSA, 1 h, RT) followed by a TRITC-conjugated secondary antibody ( 1/100, 1 h, RT). Finally, sections were imaged with a Nikon Eclipse E400 microscope equipped with a Spot Insight 3.2.0 camera and Spot 5.0 imaging software (Spot Imaging, Sterling Heights, Mich.). For tumors, mice received 75 mg/kg of pimonidazole in PBS 1 h before sacrifice. [28] Tumor sections were then probed with HP-1 antibody followed by a TRITC-conjugated secondary anti mouse antibody and Hoechst before imaging.

Spheroid Culture and Distribution of Rhodamine-Labeled Copolymers and siRNA in Spheroids.

NCI-ADR-RES spheroids of ˜500 μm were formed by liquid overlay as in [27]. Penetration of DY 547-labeled siRNA complexed with either PEG-Azo-PEI-DOPE or PEG-PEI-DOPE (200 nM, N/P 60) or of rhodamine-labeled PEG-Azo-PEI-DOPE and rhodamine-labeled PEG-PEI-DOPE (230 nM) was evaluated by confocal microscopy after 4 h incubation in media 7% FBS using Z-stack imaging with 10 μm intervals. [27] Fluorescence intensities of optical sections were quantitated using Image J software.

GFP Down-Regulation in A2780/GFP and NCI-ADR-RES/GFP Cells.

GFP down-regulation was evaluated by flow cytometry at a final siRNA concentration of 150 nM. A2780/GFP and NCI-ADR-RES/GFP cells were seeded in 24-well plates at a density of 3.5×10⁴ cells/well the day before transfection. Polyplexes were prepared with anti-GFP siRNA or negative control siRNA at an N/P ratio of 60 and added to cells in 200 μL of complete media. After 4 h, 500 μL of complete media were added, and the cells were incubated for an additional 44 h. Lipofectamine 2000 was used as a positive control. The GFP down-regulation was assessed by flow cytometry (λₑₓ 488 nm, λₑₘ 530/30 nm). Lipofectamine 2000 was used as a positive control.

Biodistribution of PEG-Azo-Rh-PEI-DOPE by Flow Cytometry.

Animal studies were approved by the Institutional Animal Care and Use Committee of Northeastern University. B16F10 tumors were implanted in 6-8 week old male C57/B6 mice (The Jackson Laboratory, Bar Harbor, Me.) by intradermal injection of 1.0×10⁶ B16F10 cells in 100 μL PBS. Tumor volumes were measured twice a week and tumor volumes calculated as volume=(width 2×length)/2. Mice bearing tumors of approximately 500 mm³ were injected intravenously with 200 μL of PBS or rhodamine B-labeled copolymers (1 mg/kg). Mice were sacrificed 4 h after injection and tumor, liver, lungs, spleen, heart and kidneys were harvested and separated into two fractions, one for fixation and sectioning, and the other for flow cytometry analysis. For sectioning, tissues were embedded in O.C.T. freezing medium and stored at −80° C. until sectioning at 5 μm thickness with a Microm HM 550 cryomicrotome (Thermo Scientific, Waltham, Mass.). Sections were counterstained with Hoechst 33342 prior to imaging by confocal microscopy. Tissue homogenates for flow cytometry were prepared by mincing tissues into small fragments which were digested with collagenase D for 30 min at 37° C. [29] Live cell FSC/SSC (200,000 gated events) were analyzed by flow cytometry immediately after dissociation. This procedure allowed obtainment of single cell suspensions as evidenced by forward and side scatter results (data not shown).

In Vivo Silencing.

Animal studies were approved by the Institutional Animal Care and Use Committee of Northeastern University. A2780/GFP tumors were implanted in 6-8 week old female nu/nu mice (The Jackson Laboratory, Bar Harbor, Me.) by subcutaneous injection of 4.0×10⁶ A2780/GFP cells in 100 μL PBS containing Matrigel (1:1 ratio). Tumors of approximately 200 mm³ were used for silencing experiments. Tumors were imaged ex vivo 48 h after intravenous administration of polyplexes formed with anti-GFP siRNA or Silencer® Negative control #5 siRNA (Ambion) in 200 μL PBS at a 1.5 mg/kg dose with a Kodak FX Imaging Station (Rochester, N.Y.). GFP fluorescence was quantitated using Image J. Tumors were processed for evaluation of GFP down-regulation on tumor homogenates by flow cytometry as described above.

Alanine Aminotransferase and Aspartate Aminotransferase Assays.

Blood form the PBS or PAPD/siGFP treated mice used for the in vivo silencing experiment was collected at 48 h in heparinized tubes before determination of serum levels of alanine aminotransferase (ALT) and aspartate aminotransferase (AST). ALT and ST were evaluated using a kit form Biomedical Research Service & Clinical Application (University at Buffalo, Buffalo, N.Y.) following manufacturer's protocol.

### Example 2

### Proposed Mechanism of Internalization of siRNA in Hypoxic Environment

The potency of the azobenzene unit for siRNA delivery was evaluated by linking azobenzene to PEG2000 at one end and to a PEI(1.8 kDa)-DOPE conjugate on the other end to obtain PAPD (FIG. 2).

PEG2000 was used as the hydrophilic block and for imparting stability in circulation. [8b, 10] The PEI-DOPE conjugate was introduced for siRNA complexation and to promote formation of micellar nanoparticles. [11] The hypoxia-sensitive polymer PAPD and its insensitive PEG-PEI-DOPE (PPD) counterpart were synthesized (FIG. 3 and data not shown) and expected to condense siRNA into nanoparticles with a PEG layer to protect it from the nuclease attack and impart stability in physiological fluids (FIG. 2). [7b, 8d, 10] The PEG groups would be detached from PAPD/siRNA complexes in the hypoxic and reductive [1b, 12] tumor environment because of degradation of the azobenzene linker; as a result PEI's positive charge would be exposed and the remaining PEI-DOPE/siRNA complexes would be taken up in the cell. [2c, 8e, 11b]

### Example 3

### siRNA Binding and Cytotoxicity

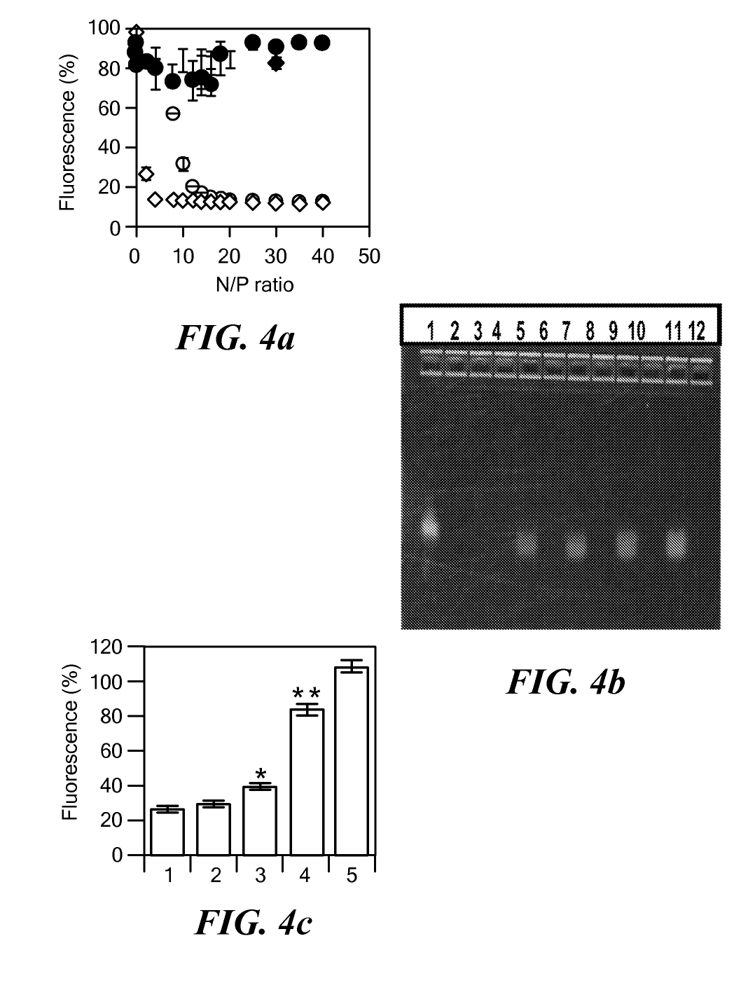

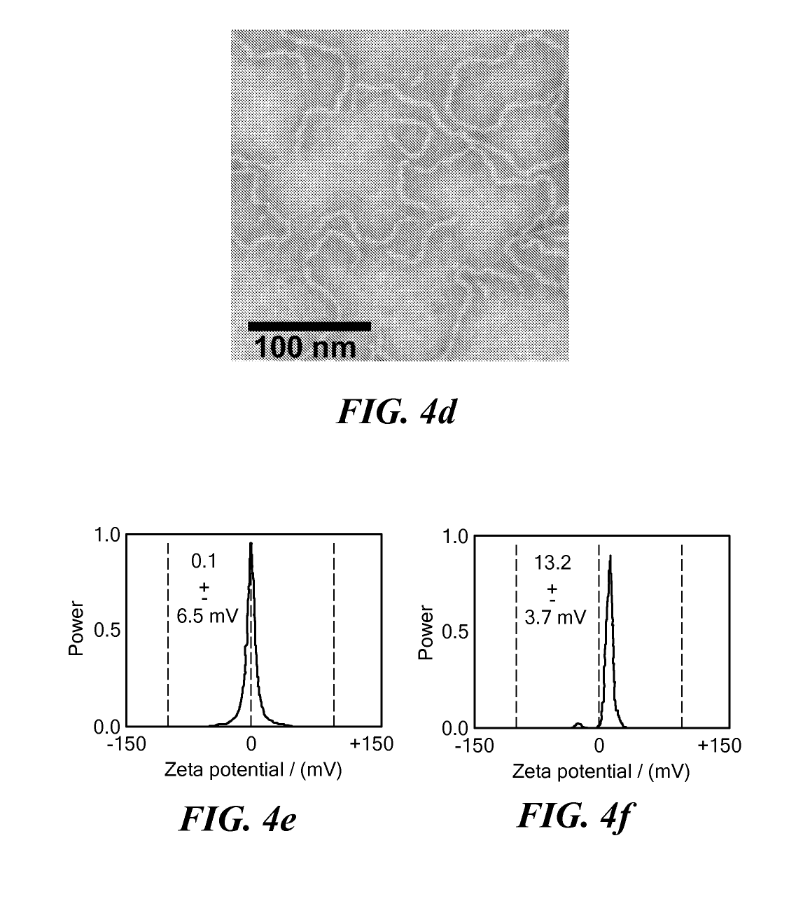

Formation of complexes between PAPD and siRNA was demonstrated by an ethidium bromide (EtBr) exclusion assay and transmission electron microscopy (FIG. 4a, 4d). In line with previous results, [13] a higher N/P ratio of PAPD over PEI was required to quench siRNA fluorescence (16 and 4, respectively). Complexes protected siRNA against RNAse degradation (FIG. 4b) and demonstrated moderate unpacking (30% increase in EtBr fluorescence, FIG. 4c) after incubation in the medium containing 10% fetal bovine serum, in agreement with Refs. [7b, 8e, 13, 14].

### Example 4

### siRNA Internalization in Monolayers and Distribution in Spheroids

Since reductase-rich rat liver microsomes were reported to cleave nitroimidazole derivatives under hypoxia, [4a,b, 9] siRNA condensation and uptake of the complexes after incubation with rat liver microsomes were evaluated (FIG. 4c). While siRNA fluorescence was quenched in PBS (26% of siRNA fluorescence), the incubation with microsomes led to a threefold increase in fluorescence (FIG. 4c) and a threefold increase in aniline absorbance (data not shown), supporting bioreductive cleavage. [4b, 12] Addition of microsomes also led to a considerable positive charge increase from (0.1±6.5) mV to (13.2±3.7) mV (p=0.006, Student's t test) (FIG. 4e, 4f). Exposure of positive surface charges from the siRNA complexes, which were previously hidden by PEG, under reductive hypoxia conditions indicates PEG detachment after azobenzene cleavage. [2c, 4a, 8a] By contrast, no such charge exposure was observed for PPD/siRNA complexes (data not shown). No cytotoxicity was detected after the cells were treated with PPD and PAPD both free and complexed with siRNA and both in normoxic and hypoxic conditions (data not shown).

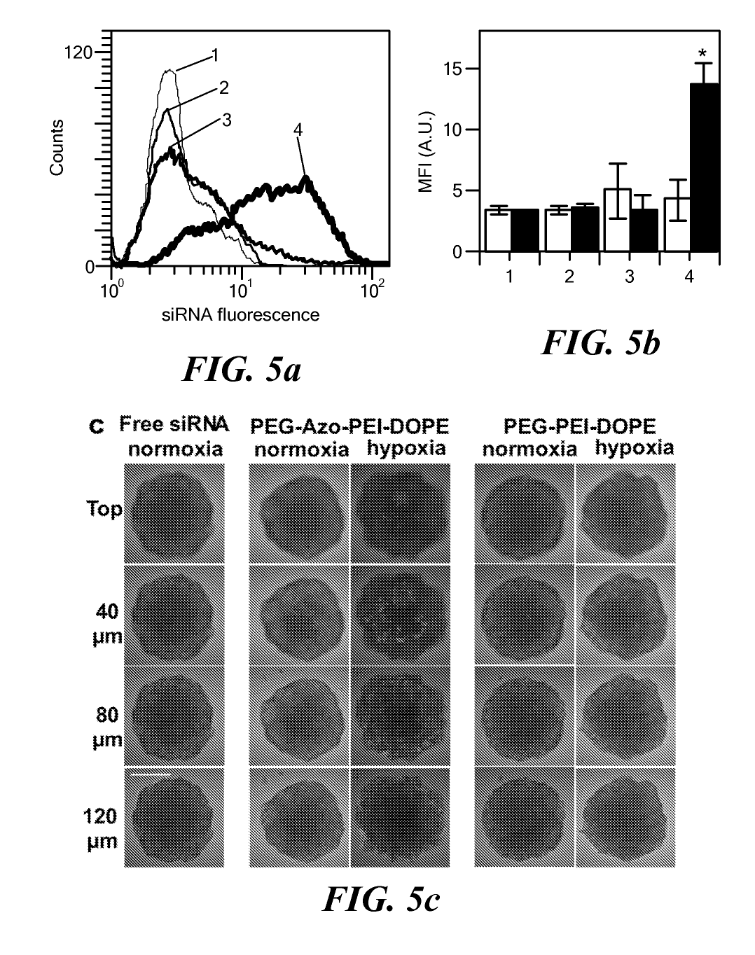

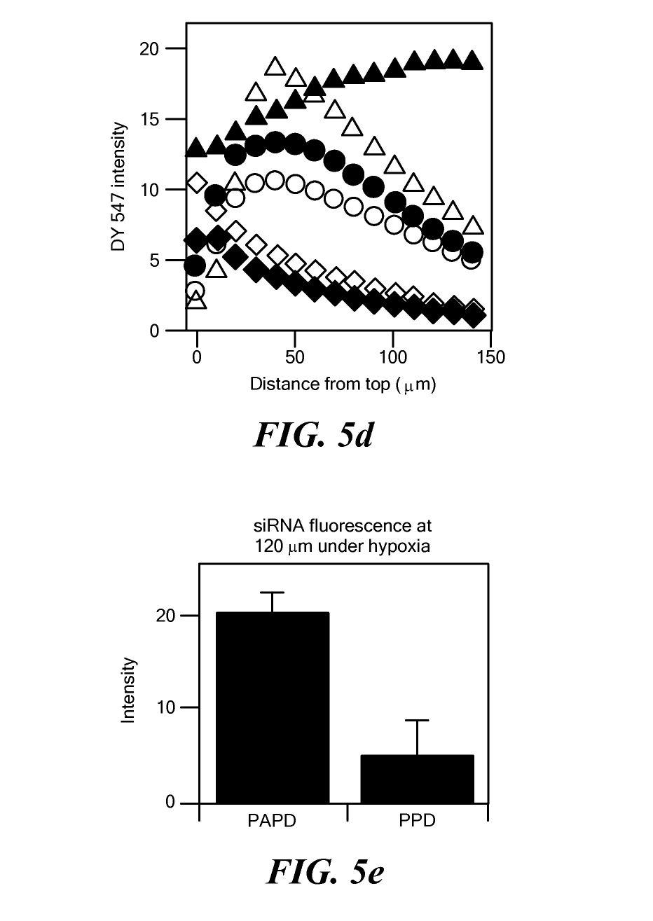

The uptake of the nanopreparations with monolayer cultures of cancer cells in normoxic and hypoxic environments was studied. In vitro hypoxia was confirmed by Hydroxyprobe staining (data not shown). [4c] Cellular internalization of PPD or PAPD complexes prepared with Fluorescein amidite (FAM)-labeled siRNA was determined by flow cytometry (FIG. 5a, 5b). The cell-associated fluorescence of PAPD/siRNA-treated cells under hypoxia was 3.2-fold higher than under normoxia (13.4 and 4.1, respectively; FIG. 5B) and 3.9-fold higher than for PPD/siRNA under hypoxia.

Cancer cell spheroids have been proposed as models for the evaluation of nanomedicines, [15] and used a spheroid model was used to confirm hypoxia-activated siRNA internalization. Whereas free FAM-siRNA fluorescence was bound to the surface of the spheroids, complexation with PAPD or PPD nanocarriers increased its penetration under normoxia, although only to the first cell layers (FIG. 5c, 5d, 5e), as reported by Wong et al. with siRNA lipoplexes. [16] Only treatment of spheroids with PAPD/siRNA under hypoxia (FIG. 5E) resulted in further increase of siRNA penetration. This was matched with a deeper penetration of rhodamine-labeled PEG-Azo-Rhodamine-PEI-DOPE (PARPD) over PEG-Rhodamine-PEI-DOPE (PRPD) (data not shown). Altogether, this data suggests better uptake of the nanoformulation after PEG deshielding. [2c, 8a,d,e]

### Example 5

### siRNA-Mediated Down Regulation in Cultured Cells

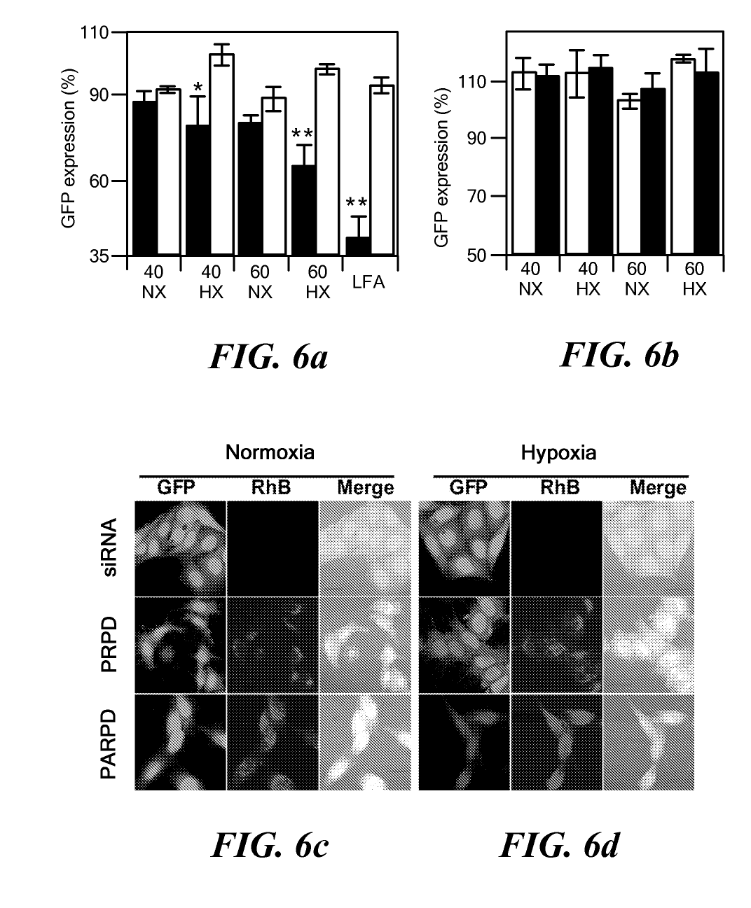

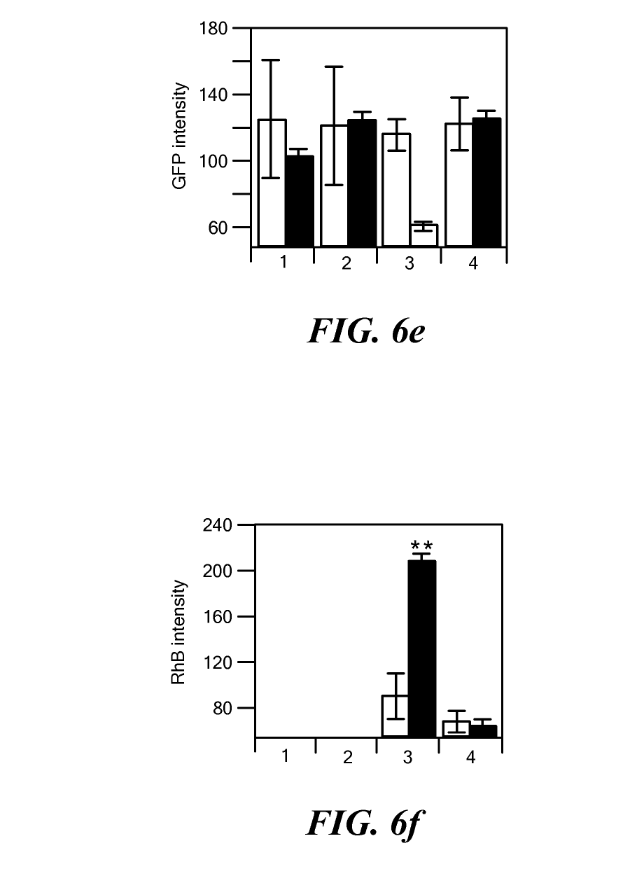

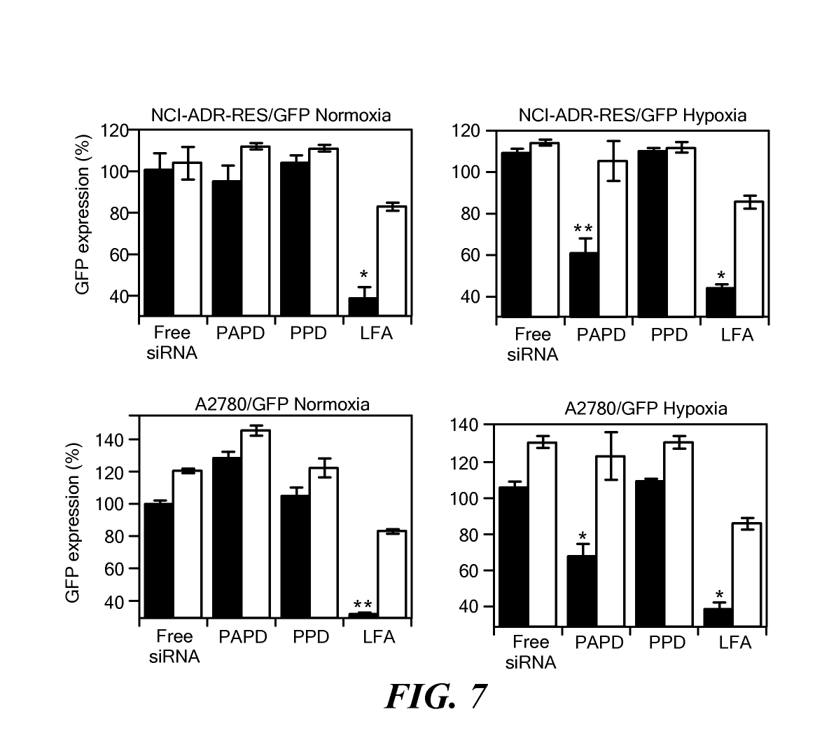

HeLa cells stably expressing GFP (HeLa/GFP) NCI-ADR-RES/GFP, and A2780/GFP cells were used to confirm the PAPD-mediated gene down regulation in the presence of 10% FBS. Whereas no GFP down regulation was observed with PAPD-complexed siRNA under normoxia (FIG. 6A), 30-40% down regulation was detected under hypoxia in all HeLa/GFP, NCI-ADR-RES/GFP, and A2780/GFP cells (FIG. 6a, FIG. 7) there was no significant down regulation when insensitive PPD/siRNA polyplexes were used (FIG. 6b). This silencing activity is comparable with that reported earlier using 200 nm siRNA. [16] Hypoxia-induced silencing was concordant with the internalization results. The silencing activity observed in vitro, which was moderate compared to that associated with Lipofectamine-mediated delivery, may be attributed to incomplete cleavage of the azobenzene unit within the observation time; however, one has to note that the silencing of Lipofectamine complexes is identical under both normoxia and hypoxia, which clearly supports the hypoxic selectivity of the azobenzene-based nanocarrier. To corroborate down regulation in hypoxic conditions with internalization, polyplexes were formed using Rhodamine B labeled copolymers (FIG. 6c-f). Stronger GFP down regulation under hypoxia over normoxia was proportional to the enhanced PARPD uptake (FIG. 6c,6d) while an opposite correlation was observed for PRPD (FIG. 6d-f).

### Example 5

### siRNA-Mediated Down-Regulation in Tumor Cells In Vivo

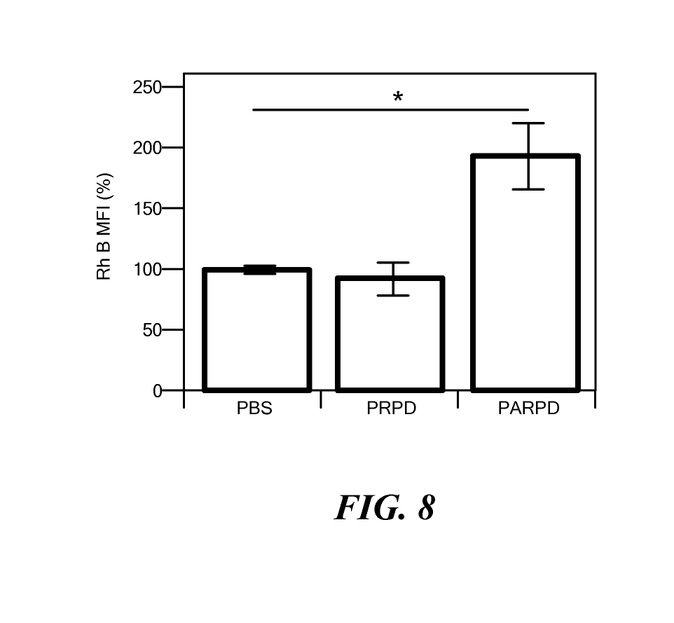

Accumulation of the copolymers in tumors 4 h after intravenous administration of PARPD and PRPD to mice bearing hypoxic B16F10 tumors was studied. [3] A twofold increase in tumor-cell associated fluorescence intensity was observed only for PARPD (FIG. 8). Fluorescence from polymers was also detected in the blood-filtering organs liver, spleen, and kidney (not shown), off-target sites for nanomedicines. [2c, 5a] Whereas PARPD was detected in tumor sections, PRPD was not found to accumulate in tumors. The data support tumor hypoxia-induced PEG shedding with subsequent PARPD uptake upon the charge exposure, in good agreement with reports on tumor-specific charge exposure. [2c, 8a,d,e]

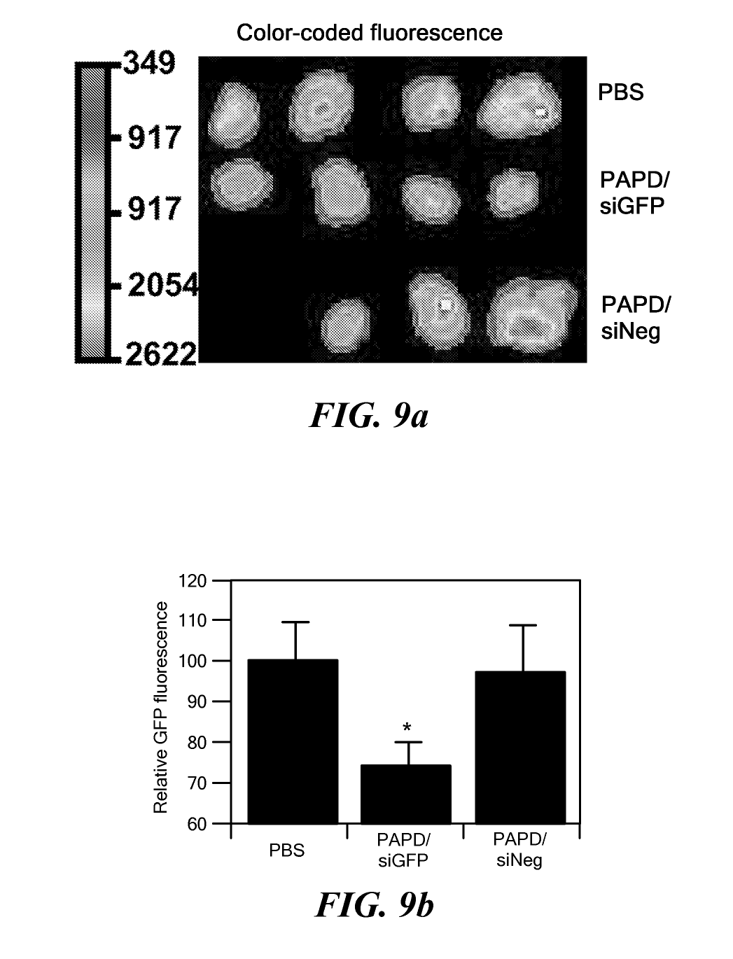

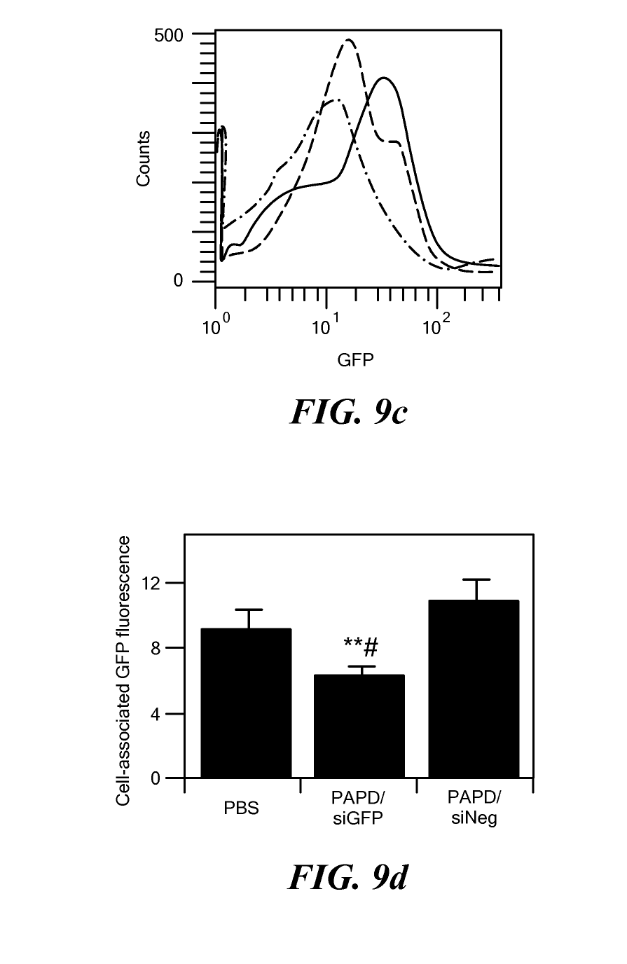

Gene silencing in vivo on A2780/GFP tumors in mice was analyzed. Substantial GFP down regulation was detected after intravenous injection of PAPD/siRNA nanoparticles both by ex vivo imaging (24%, FIG. 9a) and by flow cytometry (32%, FIG. 9b). The ex vivo imaging shows that GFP expression is highest near the center of the tumor and that GFP expression at the center of the tumors from the PBS and PAPD/siNeg-treated cells is higher than in the tumors from the PAPD/siGFP-treated cells. No downregulation was observed with PAPD complexes formed with scrambled siRNA (FIG. 9a-d). The in vivo silencing capacity of PAPD corresponded well to its in vitro uptake and silencing profiles and tumor accumulation.

As used herein, “consisting essentially of” does not exclude materials or steps that do not materially affect the basic and novel characteristics of the claim. Any recitation herein of the term “comprising”, particularly in a description of components of a composition or in a description of elements of a device, can be exchanged with “consisting essentially of” or “consisting of”.

### REFERENCES

1a. J. M. Brown, W. R. Wilson, Nat. Rev. Cancer 2004, 4, 437-447.
1b. W. R. Wilson, M. P. Hay, Nat. Rev. Cancer 2011, 11, 393-410.
2a. J. Kling, Nat. Biotechnol. 2012, 30, 381.
2b. Q. Lin, C. Bao, Y. Yang, Q. Liang, D. Zhang, S. Cheng, L. Zhu, Adv. Mater. 2013, 25, 1981-1986.
2c. Z. Poon, D. Chang, X. Zhao, P. T. Hammond, ACS Nano 2011, 5, 4284-4292.
3. C. Kieda, B. El Hafny-Rahbi, G. Collet, N. Lamerant-Fayel, C. Grillon, A. Guichard, J. Dulak, A. Jozkowicz, J. Kotlinowski, K. C. Fylaktakidou, A. Vidal, P. Auzeloux, E. Miot-Noirault, J. C. Beloeil, J. M. Lehn, C. Nicolau, J. Mol. Med. 2013, 91, 883-899.
4a. K. Kiyose, K. Hanaoka, D. Oushiki, T. Nakamura, M. Kajimura, M. Suematsu, H. Nishimatsu, T. Yamane, T. Terai, Y. Hirata, T. Nagano, J. Am. Chem. Soc. 2010, 132, 15846-15848.
4b. S. Kizaka-Kondoh, H. Konse-Nagasawa, Cancer Sci. 2009, 100, 1366-1373; c. K. A. Krohn, J. M. Link, R. P. Mason, J. Nucl. Med. 2008, 49, 129S-148S.
5a. L. Huang, Y. Liu, Annu. Rev. Biomed. Eng. 2011, 13, 507-530.
5b. D. H. Kim, J. J. Rossi, Nat. Rev. Genet. 2007, 8, 173-184.
5c. G. J. Weiss, J. Chao, J. D. Neidhart, R. K. Ramanathan, D. Bassett, J. A. Neidhart, C. H. Choi, W. Chow, V. Chung, S. J. Forman, E. Garmey, J. Hwang, D. L. Kalinoski, M. Koczywas, J. Longmate, R. J. Melton, R. Morgan, J. Oliver, J. J. Peterkin, J. L. Ryan, T. Schluep, T. W. Synold, P. Twardowski, M. E. Davis, Y. Yen, Invest. New Drugs 2013, 31, 986-1000.
6. H. Maeda, H. Nakamura, J. Fang, Adv. Drug Delivery Rev. 2013, 65, 71-79.
7a. Y. H. Bae, K. Park, J. Controlled Release 2011, 153, 198-205.
7b. M. Ogris, S. Brunner, S. Schuller, R. Kircheis, E. Wagner, Gene Ther. 1999, 6, 595-605.
8a. J. J. Li, Z. S. Ge, S. Y. Liu, Chem. Commun 2013, 49, 6974-6976.
8b. H. Hatakeyama, H. Akita, K. Kogure, M. Oishi, Y. Nagasaki, Y. Kihira, M. Ueno, H. Kobayashi, H. Kikuchi, H. Harashima, Gene Ther. 2007, 14, 68-77.
8c. T. Ishida, M. J. Kirchmeier, E. H. Moase, S. Zalipsky, T. M. Allen, Biochim. Biophys. Acta Biomembr. 2001, 1515, 144-158.
8d. S. Takae, K. Miyata, M. Oba, T. Ishii, N. Nishiyama, K. Itaka, Y. Yamasaki, H. Koyama, K. Kataoka, J. Am. Chem. Soc. 2008, 130, 6001-6009.
8e. X. Z. Yang, J. Z. Du, S. Dou, C. Q. Mao, H. Y. Long, J. Wang, ACS Nano 2012, 6, 771-781.
9. W. Piao, S. Tsuda, Y. Tanaka, S. Maeda, F. Liu, S. Takahashi, Y. Kushida, T. Komatsu, T. Ueno, T. Terai, T. Nakazawa, M. Uchiyama, K. Morokuma, T. Nagano, K. Hanaoka, Angew. Chem. 2013, 125, 13266-13270; Angew. Chem. Int. Ed. 2013, 52, 13028-13032.
10. A. L. Klibanov, K. Maruyama, A. M. Beckerleg, V. P. Torchilin, L. Huang, Biochim. Biophys. Acta Biomembr. 1991, 1062, 142-148.
11a. G. Navarro, R. R. Sawant, S. Biswas, S. Essex, C. Tros de Ilarduya, V. P. Torchilin, Nanomedicine 2012, 7, 65-78.
11b. R. R. Sawant, S. K. Sriraman, G. Navarro, S. Biswas, R. A. Dalvi, V. P. Torchilin, Biomaterials 2012, 33, 3942-3951.
11c. G. Navarro, S. Essex, R. R. Sawant, S. Biswas, D. Nagesha, S. Sridhar, C. T. de ILarduya, V. P. Torchilin, Nanomedicine: Nanotechnology, Biology and Medicine 2013.
12. H. Joo Jung, T. Geol Lee, T. Dong Chung, Chem. Commun 2010, 46, 3863-3865.
13. A. Malek, F. Czubayko, A. Aigner, J. Drug Targeting 2008, 16, 124-139.
14. R. S. Burke, S. H. Pun, Bioconjugate Chem. 2008, 19, 693-704.
15. G. Mehta, A. Y. Hsiao, M. Ingram, G. D. Luker, S. Takayama, J. Controlled Release 2012, 164, 192-204.
16. H. L. Wong, Z. Shen, Z. Lu, M. G. Wientjes, J. L. Au, Mol. Pharm. 2011, 8, 833-840.
17. M. Omedes Pujol, D. J. Coleman, C. D. Allen, O. Heidenreich, D. A. Fulton, J. Controlled Release 2013, 172, 939-945.
18. S. Biswas, P. P. Deshpande, G. Navarro, N. S. Dodwadkar, V. P. Torchilin, Biomaterials 2013, 34, 1289-1301.
19. F. Perche, D. Gosset, M. Mevel, M. L. Miramon, J. J. Yaouanc, C. Pichon, T. Benvegnu, P. A. Jaffres, P. Midoux, J Drug Target 2011, 19, 315-325.
20. A. A. Beharry, G. A. Woolley, Chem Soc Rev 2011, 40, 4422-4437.
21. Q. Lin, C. Bao, Y. Yang, Q. Liang, D. Zhang, S. Cheng, L. Zhu, Adv Mater 2013, 25, 1981-1986.
22. S. Iwaki, K. Hanaoka, W. Piao, T. Komatsu, T. Ueno, T. Terai, T. Nagano, Bioorg Med Chem Lett 2012, 22, 2798-2802.
23a. A. V. Caramyshev, E. G. Evtushenko, V. F. Ivanov, A. R. Barcelo, M. G. Roig, V. L. Shnyrov, R. B. van Huystee, I. N. Kurochkin, A. Vorobiev, I. Y. Sakharov, Biomacromolecules 2005, 6, 1360-1366.
23b. H. JooáJung, T. GeoláLee, T. DongáChung, Chem Comm 2010, 46, 3863-3865.
24. a J. DeRouchey, G. F. Walker, E. Wagner, J. O. Radler, The Journal of Physical Chemistry B 2006, 110, 4548-4554.
24b. K. Osada, H. Oshima, D. Kobayashi, M. Doi, M. Enoki, Y. Yamasaki, K. Kataoka, Journal of the American Chemical Society 2010, 132, 12343-12348; c J. DeRouchey, C. Schmidt, G. F. Walker, C. Koch, C. Plank, E. Wagner, J. O. Radler, Biomacromolecules 2008, 9, 724-732.
25. K. A. Krohn, J. M. Link, R. P. Mason, J Nucl Med 2008, 49 Suppl 2, 129S-148S.
26. J. Tupper, O. Greco, G. M. Tozer, G. U. Dachs, Cancer Gene Therapy 2004, 11, 508-513.
27. F. Perche, N. R. Patel, V. P. Torchilin, J Control Release 2012, 164, 95-102.
28. C. Kieda, B. El Hafny-Rahbi, G. Collet, N. Lamerant-Fayel, C. Grillon, A. Guichard, J. Dulak, A. Jozkowicz, J. Kotlinowski, K. C. Fylaktakidou, A. Vidal, P. Auzeloux, E. Miot-Noirault, J. C. Beloeil, J. M. Lehn, C. Nicolau, J Mol Med (Berl) 2013, 91, 883-899.
29. M. Kortylewski, M. Kujawski, T. Wang, S. Wei, S. Zhang, S. Pilon-Thomas, G. Niu, H. Kay, J. Mule, W. G. Kerr, R. Jove, D. Pardoll, H. Yu, Nat Med 2005, 11, 1314-1321.

What is claimed is:

1. A hypoxia-sensitive polynucleotide-binding molecule comprising:
(1) an uncharged hydrophilic polymer;
(2) an azobenzene moiety, wherein the azobenzene moiety is attached to the to the uncharged hydrophilic polymer by a first covalent linkage;
(3) a positively-charged polymer, wherein the positively-charged polymer is attached to the azobenzene moiety by a second covalent linkage, and wherein the positively-charged polymer binds one or more polynucleotide molecules; and
(4) a phospholipid, wherein the phospholipid is attached to the positively-charged polymer by a third covalent linkage;
wherein the uncharged hydrophilic polymer, the azobenzene moiety, the positively-charged polymer, and the phospholipid are present in the molecule in about a 1:1:1:1 molar ratio.

2. The molecule of claim 1, wherein the uncharged polymer is selected from the group consisting of polyethylene glycol, polyvinylpyrrolidone, and polyacrylamide.

3. The molecule of claim 2, wherein the uncharged polymer is polyethylene glycol

4. The molecule of claim 3, wherein the polyethlylene glycol has an average molecular weight from about 1000 to about 5000 daltons.

5. The molecule of claim 4, wherein the polyethlylene glycol has an average molecular weight of about 2000 daltons.

6. The molecule of claim 1, wherein the azobenzene moiety is azobenzene-4,4′-dicarboxamide.

7. The molecule of claim 1, wherein the positively-charged polymer is selected from the group consisting of polyethylenimine, polylysine, a cationic peptide, poly(dl-lactide-co-glycolide), poly(amidoamine), and poly(propylenimine).

8. The molecule of claim 7, wherein the positively-charged polymer is polyethylenimine.

9. The molecule of claim 8, wherein the polyethylenimine has a molecular weight from about 500 daltons to about 5000 daltons.

10. The molecule of claim 9, wherein the polyethylenimine is has an average molecular weight of about 1800 daltons.

11. The molecule of claim 8, wherein the polyethylenimine has a branched structure.

12. The molecule of claim 1, wherein the phospholipid is selected from the group consisting of phosphatidic acid, phosphatidylethanolamine, phosphatidylcholine, phosphatidylserine, phosphatidylinositol, phosphotidylglycerol, and a sphingolipid.

13. The molecule of claim 12, wherein the phospholipid comprises fatty acid side chains each having from 12-20 carbon atoms.

14. The molecule of claim 13, wherein the fatty acid side chains are saturated, monounsaturated, diunsaturated, or triunsaturated.

15. The molecule of claim 12, wherein the phospholipid is phosphtatidylethanolamine.

16. The molecule of claim 15, wherein the phosphatidylethanolamine is 1,2-dioleoyl-sn-glycero-3-phosphoethanolamine.

17. The molecule of claim 1, wherein each of the first, second, and third covalent linkages is independently selected from the group consisting of a peptide bond, amide bond, ester bond, ether bond, alkyl bond, carbonyl bond, alkenyl bond, thioether bond, azide bond, and disulfide bond.

18. The molecule of claim 1, wherein the first, second, and third covalent linkages are peptide bonds.

19. A nanoparticle composition for delivery of a polynucleotide to a hypoxic cell or tissue, the composition comprising a plurality of molecules of claim 1 suspended in an aqueous medium and aggregated to form one or more nanoparticles.

20. The nanoparticle composition of claim 19, wherein one or more polynucleotides are non-covalently bound to the positively-charged polymers of said molecule.

21. The nanoparticle composition of claim 20, wherein the one or more polynucleotides are selected from the group consisting of single-stranded RNA, double-stranded RNA, single-stranded DNA, and double-stranded RNA.

22. The nanoparticle composition of claim 21, wherein the one or more polynucleotides are siRNA.

23. The nanoparticle of composition of claim 22, wherein the composition has two or more polynucleotides, and wherein the polynucleotides are two or more different species of siRNA.

24. The nanoparticle composition of claim 20, wherein the polynucleotide is an antisense oligonucleotide.

25. The nanoparticle composition of claim 20, wherein the polynucleotide targets the expression of one or more genes selected from the group consisting of survivin, Eg5, EGFR, XIAP, CDC45L, SUV420h1, WEE1, HDAC2, RBX 1, CDK4, CSN5, FOXM1, R1 (RAM2), LSD1, CSTF2, Nectin-4, ERCC6L, PKIB, NAALADL2, PRMT1, COPZ1, SYNGR4, P-glycoprotein, VEGFR, and VEGF.

26. The nanoparticle composition of 20, wherein the nanoparticle composition has a nitrogen:phosphate ratio from about 1:5 to about 1:50.

27. The nanoparticle composition of claim 19, wherein the nanoparticles are micelles.

28. The nanoparticle composition of claim 27, wherein the micelles have a worm-like morphology.

29. The nanoparticle composition of claim 27, wherein the micelles have an average diameter from about 10 to about 50 nm.

30. The nanoparticle composition of claim 19, wherein the hypoxic cell or tissue is associated with cancer.

31. The nanoparticle composition of claim 30, wherein the cancer is associated with a solid tumor.

32. The nanoparticle composition of claim 30, wherein the cancer is selected from the group consisting of uterine cancer, cervical cancer, prostate cancer, ovarian cancer, sarcoma, and head and neck cancer.

33. The nanoparticle composition of claim 19, wherein the azobenzene moiety of said molecules is cleavable in a hypoxic environment.

34. The nanoparticle composition of 33, wherein cleavage of the azobenzene moiety causes release of the uncharged hydrophilic polymers from the nanoparticles.

35. The nanoparticle composition of claim 20, wherein the azobenzene moiety of said molecules is cleavable in a hypoxic environment, and said cleavage results in increased cellular uptake of said bound polynucleotides.

36. The nanoparticle composition of claim 19, further comprising a hydrophobic pharmaceutical agent.

37. The nanoparticle composition of claim 36, wherein the hydrophobic pharmaceutical agent is an anti-cancer agent.

38. The nanoparticle composition of claim 37, wherein the anti-cancer agent is selected from the group consisting of altretamine, aminoglutethimide, amsacrine (m-AMSA), azacitidine, baccatin III, bleomycin, busulfan, carmustine (BCNU), chlorambucil, cytarabine HCl, dacarbazine, dactinomycin, daunorubicin, docetaxel, doxorubicin, etoposide (VP-16), 5-fluorouracil, floxuridine, flutamide, hydroxyurea, ifosfamide, leuprolide acetate, lomustine (CCNU), melphalan, methotrexate, mitomycin, mitotane (o.p'-DDD), octreotide, paclitaxel, pentostatin, plicamycin, procarbazine HCl, semustine (methyl-CCNU), streptozocin, tamoxifen citrate, teniposide (VM-26), thioguanine, thiotepa, vindesine, vinblastine, and vincristine sulfate.

39. The nanoparticle composition of claim 19, wherein the composition consists of a plurality of said hypoxia-sensitive polynucleotide-binding molecules.

40. A pharmaceutical composition comprising the nanoparticle composition of claim 19 suspended in an aqueous buffer.

41. A pharmaceutical composition comprising the nanoparticle composition of claim 20 suspended in an aqueous buffer.

42. The pharmaceutical composition of any one of claims 40 and 41, further comprising an excipient.

43. A method of making the hypoxia-sensitive polynucleotide-binding molecule of claim 1 from the uncharged polymer having a first reactive group, an azobenzene derivative having a second and a third reactive group, the positively-charged polymer having a fourth and a fifth reactive group, and the phospholipid having a sixth reactive group, the method comprising the steps of:
(1) reacting the first reactive group on the uncharged hydrophilic polymer with the second reactive group on the azobenzene derivative, wherein the uncharged hydrophilic polymer and the azobenzene derivative are present in about a 1:1 molar ratio, to create the first covalent linkage;
(2) reacting the third reactive group on the azobenzene derivative with the fourth reactive group on the positively-charged polymer, wherein the azobenzene derivative and the positively charged polymer are present in about a 1:1 molar ratio, to create the second covalent linkage; and
(3) reacting the fifth reactive group on the positively-charged polymer with the sixth reactive group on the phospholipid, wherein the positively-charged polymer and the phospholipid are present in about a 1:1 molar ratio, to create the third covalent linkage.

44. The method of claim 43, wherein the steps are performed in the following order: (1), (2), and (3).

45. The method of claim 43, wherein the steps are performed in the following order: (1), (3), and (2).

46. The method of claim 43, wherein the steps are performed in the following order: (2), (1), and (3).

47. The method of claim 43, wherein the steps are performed in the following order: (2), (3), and (1).

48. The method of claim 43, wherein the steps are performed in the following order: (3), (1), and (2).

49. The method of claim 43, wherein the steps are performed in the following order: (3), (2), and (1).

50. The method of any one of claims 43 to 49, wherein the hydrophilic polymer is polyethylene glycol.

51. The method of claim 50, wherein the polyethlylene glycol has an average molecular weight from about 1000 to about 5000 daltons.

52. The method of claim 51, wherein the polyethylene glycol is polyethylene glycol 2000-N-hydroxysuccinamide ester.

53. The method of any one of claims 43 to 49, wherein the positively-charged polymer is polyethylenimine.

54. The method of claim 53, wherein the polyethylenimine has a molecular weight from about 500 daltons to about 5000 daltons.

55. The method of claim 54, wherein the polyethylenimine is branched and has an average molecular weight of about 1800 daltons.

56. The method of any one of claims 43 to 49, wherein the phospholipid is phosphtatidylethanolamine.

57. The method of claim 56, wherein the phosphatidylethanolamine is 1,2-dioleoyl-sn-glycero-3-phosphoethanolamine-N-(glutaryl).

58. The method of any one of claims 43 to 49, wherein the azobenzene derivative is azobenzene-4,4′-dicarboxylic acid.

59. The method of any one of claims 43 to 49, wherein each of the first, second, and third covalent linkages is independently selected from the group consisting of a peptide bond, amide bond, ester bond, ether bond, alkyl bond, carbonyl bond, alkenyl bond, thioether bond, disulfide bond, and azide bond.

60. The method of claim 59, wherein the first, second, and third covalent linkages are peptide bonds.

61. The method of claim 43, wherein the hydrophilic polymer is polyethylene glycol 2000-N-hydroxysuccinamide ester, the azobenzene derivative is azobenzene-4,4′-dicarboxylic acid, the positively-charged polymer is branched polyethylenimine having an average molecular weight of about 1800 daltons, and the phospholipid is 1,2-dioleoyl-sn-glycero-3-phosphoethanolamine-N-(glutaryl).

62. The method of claim 61, wherein:
step (1) is performed in the presence of N-(3-dimethylaminopropyl)N′-ethylcarbodiimide hydrochloride, N-hydroxysuccinimide, pyridine, and 4-dimethylaminopyridine;
step (2) is performed in the presence of N-(3-dimethylaminopropyl)N′-ethylcarbodiimide hydrochloride, N-hydroxysuccinimide, triethylamine, and CHCl₃; and
step (3) is performed in the presence of N-(3-dimethylaminopropyl)N′-ethylcarbodiimide hydrochloride, N-hydroxysuccinimide, triethylamine, and CHCl₃.

63. A method of making a nanoparticle composition comprising hypoxia-sensitive polynucleotide-binding molecules of claim 1, the method comprising the steps of:
(1) providing a solution of the hypoxia-sensitive polynucleotide-binding molecules in a non-aqueous solvent; and
(2) replacing the non-aqueous solvent with an aqueous medium to form an aqueous suspension comprising nanoparticles, the nanoparticles comprising aggregates of a plurality of the hypoxia-sensitive polynucleotide-binding molecules.

64. The method of claim 63, wherein step (2) comprises dialyzing the solution of hypoxia-sensitive polynucleotide-binding molecules against an aqueous medium to form the nanoparticles.

65. The method of claim 63, wherein step (2) comprises:
(a) evaporating the non-aqueous solvent to form a dry film of the hypoxia-sensitive polynucleotide-binding molecules; and
(b) suspending the dry film in an aqueous medium to form the nanoparticles.

66. The method of claim 63, wherein the nanoparticle composition consists of a plurality of the hypoxia-sensitive polynucleotide-binding molecules.

67. The method of claim 63, further comprising the step of adding a hydrophobic pharmaceutical agent to the solution of hypoxia-sensitive polynucleotide-binding molecules in a non-aqueous solvent, wherein the nanoparticles produced by replacing the non-aqueous solvent with an aqueous medium comprise the hydrophobic pharmaceutical agent.

68. The method of claim 63, further comprising the step of adding a hydrophobic pharmaceutical agent to the aqueous suspension comprising nanoparticles, whereby the hydrophobic pharmaceutical agent is incorporated into the nanoparticles.

69. The method of claim 63, further comprising the step of adding one or more polynucleotides to the aqueous suspension comprising nanoparticles, whereby the one or more polynucleotides become non-covalently bound to the positively-charged polymers of said nanoparticles.

70. A method of treating a disease or condition associated with a hypoxic cell or tissue, the method comprising administering to a subject having or suspected of having the disease or condition the nanoparticle composition of claim 19.

71. The method of claim 70, wherein the disease or condition is cancer.

72. The method of claim 69, wherein the cancer is associated with a solid tumor.

73. The method of claim 70, wherein the cancer is selected from the group consisting of uterine cancer, cervical cancer, prostate cancer, ovarian cancer, sarcoma, and head and neck cancer.

74. The method of claim 70, wherein the nanoparticle composition is administered by a parenteral route.

75. The method of claim 74, wherein the parenteral administration route is selected from the group consisting of intravascular administration, peri- and intra-tissue administration, subcutaneous injection or deposition, subcutaneous infusion, intraocular administration, and direct application at or near a site of neovascularization.

76. The method of claim 70, wherein the nanoparticle composition comprises a hypoxia-sensitive, polynucleotide-binding molecule comprising polyethlylene glycol having an average molecular weight of about 2000 daltons, azobenzene-4,4′-dicarboxamide, polyethylenimine having an average molecular weight of about 1800 daltons, and 1,2-dioleoyl-sn-glycero-3-phosphoethanolamine.

77. The method of claim 70, wherein the nanoparticle comprises a polynucleotide.

78. The method of claim 77, wherein the polynucleotide targets the expression of one or more genes selected from the group consisting of survivin, Eg5, EGFR, XIAP, CDC45L, SUV420h1, WEE1, HDAC2, RBX 1, CDK4, CSN5, FOXM1, R1 (RAM2), LSD1, CSTF2, Nectin-4, ERCC6L, PKIB, NAALADL2, PRMT1, COPZ1, SYNGR4, P-glycoprotein, VEGFR, and VEGF.

79. The method of claim 70, wherein the nanoparticle comprises a hydrophobic pharmaceutical agent.

80. The method of claim 79, wherein the hydrophobic pharmaceutical agent is selected from the group consisting of altretamine, aminoglutethimide, amsacrine (m-AMSA), azacitidine, baccatin III, bleomycin, busulfan, carmustine (BCNU), chlorambucil, cytarabine HCl, dacarbazine, dactinomycin, daunorubicin, docetaxel, doxorubicin, etoposide (VP-16), 5-fluorouracil, floxuridine, flutamide, hydroxyurea, ifosfamide, leuprolide acetate, lomustine (CCNU), melphalan, methotrexate, mitomycin, mitotane (o.p'-DDD), octreotide, paclitaxel, pentostatin, plicamycin, procarbazine HCl, semustine (methyl-CCNU), streptozocin, tamoxifen citrate, teniposide (VM-26), thioguanine, thiotepa, vindesine, vinblastine, or vincristine sulfate.

81. A kit for use in treating a disease or condition associated with a hypoxic cell or tissue, the kit comprising:
(a) the molecule of claim 1; and
(b) packaging therefor.

82. The kit of claim 81, wherein the hypoxia-sensitive polynucleotide-binding molecules are provided as a dry powder or film.

83. The kit of claim 82, further comprising instructions for reconstituting the hypoxia-sensitive polynucleotide-binding molecules as micelles in an aqueous suspension.

84. The kit of claim 81, wherein the hypoxia-sensitive polynucleotide-binding molecules are provided in the form of an aqueous suspension comprising a plurality of nanoparticles comprising the hypoxia-sensitive polynucleotide-binding molecules.

85. The kit of claim 81, further comprising a polynucleotide.

86. The kit of claim 85, further comprising instructions for forming a nanoparticle composition comprising the hypoxia-sensitive polynucleotide-binding molecule and the polynucleotide.

87. The kit of claim 81, further comprising a hydrophobic pharmaceutical agent.

88. The kit of claim 87, further comprising instructions for forming a nanoparticle composition comprising the hypoxia-sensitive polynucleotide-binding molecule and the hydrophobic pharmaceutical agent.

89. The kit of claim 81, further comprising instructions for use of the kit.

90. A kit for use in treating a disease or condition associated with a hypoxic cell or tissue, the kit comprising:
(a) the nanoparticle composition of claim 19; and
(b) packaging therefor.

91. The kit of claim 90, further comprising a polynucleotide.

92. The kit of claim 91, further comprising instructions for forming non-covalent bonds between the polynucleotide and the nanoparticle composition.

93. A kit for treating a disease or condition associated with a hypoxic cell or tissue, the kit comprising the nanoparticle composition of claim 36.

94. The kit of claim 93, further comprising a polynucleotide.

95. The kit of claim 94, further comprising instructions for forming non-covalent bonds between the polynucleotide and the nanoparticle composition.

96. The kit of claim 90, further comprising instructions for use of the kit.

97. A kit for treating a disease or condition associated with a hypoxic cell or tissue, the kit comprising the pharmaceutical composition of claim 40.

98. The kit of claim 97, further comprising a polynucleotide.

99. The kit of claim 97, further comprising instructions for forming non-covalent bonds between the polynucleotide and the nanoparticle composition.

100. The kit of claim 97, further comprising instructions for use of the kit.
